(19) United States
(12) Patent Application Publication
(10) Pub. No.: US 2016/0175586 A1
(43) Pub. Date: Jun. 23, 2016

(54) EPIDURAL STIMULATION FOR FACILITATION OF LOCOMOTION, POSTURE, VOLUNTARY MOVEMENT, AND RECOVERY OF AUTONOMIC, SEXUAL, VASOMOTOR, AND COGNITIVE FUNCTION AFTER NEUROLOGICAL INJURY

(71) Applicants: Victor Reggie Edgerton, Los Angeles, CA (US); Roland R. Roy, Playa Vista, CA (US); Yury Gerasimenko, Los Angeles, CA (US); Joel W. Burdick, Pasadena, CA (US); Parag Gad, Woodland Hills, CA (US); Nicholas A. Terrafranca, Laguna Niguel, CA (US)

(72) Inventors: Victor Reggie Edgerton, Los Angeles, CA (US); Roland R. Roy, Playa Vista, CA (US); Yury Gerasimenko, Los Angeles, CA (US); Joel W. Burdick, Pasadena, CA (US); Parag Gad, Woodland Hills, CA (US); Nicholas A. Terrafranca, Laguna Niguel, CA (US)

(73) Assignee: NEURORECOVERY TECHNOLOGIES, INC., Laguna Niguel, CA (US)

(21) Appl. No.: 14/880,100

(22) Filed: Oct. 9, 2015

Related U.S. Application Data
(60) Provisional application No. 62/062,755, filed on Oct. 10, 2014.

(51) Int. Cl. A61N 1/36; A61N 1/05; A61N 1/04

(52) U.S. Cl. CPC A61N 1/36003; A61N 1/0452; A61N 1/0551; A61N 1/36014; A61N 1/36067

(57) ABSTRACT

Methods are described comprising: administering to a mammal with a paralysis an electrical enabling motor control stimulation to at a sub-threshold location, wherein the electrical enabling motor control stimulation provides spontaneous voluntary movement of at least one body part.

### CROSS-REFERENCE TO RELATED APPLICATIONS

The present application claims the benefit of U.S. Provisional Patent Application No. 62/062,755, filed on Oct. 10, 2014, the entire content of which is incorporated herein by reference.

### STATEMENT REGARDING FEDERALLY SPONSORED RESEARCH OR DEVELOPMENT

This invention was made with Government support under Grant No. W81XWH-09-2-0024, awarded by the United States Army, Medical Research and Materiel Command; Grant No. EB007615, awarded by the National Institute of Health; and Grant No. R01NS062009, awarded by the National Institute of Health. The Government has certain rights in this invention.

### BACKGROUND

1. Field

The present invention relates to the field neurological rehabilitation for injury and disease including traumatic spinal cord injury, non-traumatic spinal cord injury, stroke, movement disorders, brain injury, and other diseases or injuries that result in paralysis and/or nervous system disorder. Devices, pharmacological agents, and methods are provided to facilitate recovery of posture, locomotion, and voluntary movements of the arms, trunk, and legs, and recovery of autonomic, sexual, vasomotor, and cognitive function, in a human subject having spinal cord injury, brain injury, or any other neurological disorder.

2. Description of the Related Art

Serious spinal cord injuries (SCI) affect approximately 250,000 people in the United States, and roughly 11,000 new injuries occur each year. Of these injuries, approximately 50% are complete spinal cord injuries in which there is essentially total loss of sensory motor function below the level of the spinal lesion.

For chronic SCI humans, impressive levels of standing and stepping recovery has been demonstrated in certain incomplete SCI subjects with task specific physical rehabilitation training. A recent clinical trial demonstrated that 92% of the subjects regained stepping ability to almost a functional speed of walking three months after a severe yet incomplete injury (Dobkin et al., Neurology, 66(4): 484-93 (2006)) and in chronic subjects months to years after injury (Harkema et. al., Archives of Physical Medicine and Rehabilitation: 2011 epub). Furthermore, improved coordination of motor pool activation can be achieved with training in patients with incomplete SCI (Field-Fote et al., Phys. Ther., 82 (7): 707-715 (2002)). On the other hand, there is no generally accepted evidence that an individual with a clinically complete SCI can be trained to the point where they could stand or locomote even with the aid of a “walker” (Wernig, Arch Phys Med Rehabil., 86(12): 2385-238 (2005)) and no one has shown the ability to regain voluntary movements and/or to recover autonomic, sexual, vasomotor, and/or improved cognitive function after a motor complete spinal cord injury.

To date, the consistently most successful intervention for regaining weight-bearing stepping in humans is weight-bearing step training, but that has been the case primarily in subjects with incomplete injuries.

The most effective future strategies for improving motor and autonomic functions that improve the quality of life post-SCI will likely involve the combination of many different technologies and strategies, as neurological deficits such as spinal cord injuries are complex, and there is a wide variability in the deficit profile among patients. In the long run, neuro-regenerative strategies hold significant promise for functional sensory-motor recovery from traumatic and progressive neurological deficits. Progress is already being made particularly in the case of acute treatment of incomplete spinal injuries. However, even when these strategies are perfected, other remedies will be needed. It is naive to think that neuro-regenerative approaches will recover fully functional postural and locomotor function as well as voluntary control of lower limb, and voluntary upper limb movement following a motor complete spinal injury.

### SUMMARY

Embodiments are for use with a mammal such as a human patient (or subject) who has a spinal cord with at least one selected spinal circuit and a neurologically derived paralysis in a portion of the patient's body. By way of non-limiting examples, when activated, the selected spinal circuit may (a) enable voluntary movement of muscles involved in at least one of standing, stepping, reaching, grasping, voluntarily changing positions of one or both legs, voiding the patient's bladder, voiding the patient's bowel, breathing, swallowing, chewing, postural activity, and locomotor activity; (b) enable or improve autonomic control of at least one of cardiovascular function, body temperature, and metabolic processes; and/or (c) help facilitate recovery of at least one of an autonomic function, sexual function, vasomotor function, and cognitive function.

In some embodiments, methods are described comprising: administering to a mammal with a paralysis an electrical enabling motor control stimulation to at a sub-threshold location, wherein the electrical enabling motor control stimulation provides spontaneous voluntary movement of at least one body part.

The at least one body part can be, but is not limited to, a toe, ankle, leg, knee, finger, elbow, shoulder, hand, hip, chest, trunk, neck, movement of erect penis, or a combination thereof.

In some embodiments, the paralysis was caused by a neurodegenerative injury or disease, wherein the neurodegenerative injury or disease is located within the nervous system associated with at least one of Parkinson's disease, Huntington's disease, Alzheimer's, ischemia, stroke, amyotrophic lateral sclerosis (ALS), primary lateral sclerosis (PLS), dystonia, and cerebral palsy.

In some embodiments, the electrical enabling motor control stimulation can be applied by one or more electrode or electrode array(s) that are implanted epidurally in the spinal cord of the mammal.

In some embodiments, the electrical enabling motor control stimulation is applied by one or more electrode or electrode array that is applied transcutaneously over a region of the spinal cord of the mammal. In some embodiments the electrode array may be external or placed on the service of the skin. In other embodiments there maybe a combination of one or more electrodes implanted and/or placed on the skin.

The methods described can further include: administering one or more neuropharmaceutical agents to the mammal.

In some embodiments, the administration of the electrical enabling motor control stimulation makes the mammal at least 2 times more active than without the electrical enabling motor control stimulation. In other embodiments, the administration of the electrical enabling motor control stimulation makes the mammal at least 5 times more active than without the electrical enabling motor control stimulation.

The spontaneous voluntary movement described herein can be a stepping-like activity or a partial weight bearing standing activity, a reaching activity, a grasping activity, a pulling activity, a pushing activity, extending activity, a flexing activity, rotating activity, movement of a erect penis, or the like.

The methods described herein can further include administering the electrical enabling motor control stimulation in a positive environment. The positive environment can be a location familiar to the mammal. Further, the positive environment can assist in attaining spontaneous voluntary movement.

The methods described herein can further comprise: administering the electrical enabling motor control stimulation based on at least one sensory input.

The paralysis may be a motor complete paralysis or a motor incomplete paralysis. The paralysis may have been caused by a spinal cord injury classified as motor complete or motor incomplete. The paralysis may have been caused by an ischemic or traumatic brain injury. The paralysis may have been caused by an ischemic brain injury that resulted from a stroke or acute trauma. By way of another example, the paralysis may have been caused by a neurodegenerative brain injury. The neurodegenerative brain injury may be associated with at least one of Parkinson's disease, Huntington's disease, Alzheimer's, ischemia, stroke, amyotrophic lateral sclerosis (ALS), primary lateral sclerosis (PLS), and cerebral palsy.

One exemplary embodiment is a method that includes positioning the human patient in a training device. The training device is configured to assist with physical training (e.g., at least one of standing, stepping, reaching, moving one or both legs, moving one or both feet, grasping, and stabilizing sitting posture) that is configured to induce neurological signals (e.g., at least one of postural proprioceptive signals, locomotor proprioceptive signals, and supraspinal signals) in the portion of the patient's body having the paralysis. The training device may include a robot training device configured to move automatically or assist in moving at least a portion of the portion of the patient's body having the paralysis. By way of non-limiting example, the training device may include a treadmill and a weight-bearing device configured to support at least a portion of the patient's body weight when the patient is positioned to use the treadmill. By way of another non-limiting example, the training device may include a device configured to bear at least a portion of the patient's body weight when the patient transitions between sitting and standing. By way of non-limiting example the training device may include a device configured to move at least a portion of the patient's hand, arm, or upper extremity. By way of non-limiting example the training device may include a device configured to support a patient or a portion of a patient to assist them in walking and standing upright.

The selected spinal circuit has a first stimulation threshold representing a minimum amount of stimulation required to activate the selected spinal circuit, and a second stimulation threshold representing an amount of stimulation above which the selected spinal circuit is fully activated and adding the induced neurological signals has no additional effect on the at least one selected spinal circuit. The induced neurological signals are below the first stimulation threshold and insufficient to activate the at least one selected spinal circuit.

The method also includes applying electrical stimulation to a portion of a spinal cord of the patient. The electrical stimulation may be applied by an electrode array that is implanted epidurally in the spinal cord of the patient. Such an electrode array may be positioned at at least one of a lumbosacral region, a cervical region, and a thoracic region of the spinal cord or brainstem. The electrical stimulation is below the second stimulation threshold such that the at least one selected spinal circuit is at least partially activatable by the addition of at least one of (a) a second portion of the induced neurological signals, and (b) supraspinal signals. While not a requirement, the first portion of the induced neurological signals may be the same as the second portion of the induced neurological signals. While also not a requirement, the electrical stimulation may not directly activate muscle cells in the portion of the patient's body having the paralysis. The electrical stimulation may include at least one of tonic stimulation and intermittent stimulation. The electrical stimulation may include simultaneous or sequential stimulation of different parts or regions of the spinal cord.

If the paralysis was caused by a spinal cord injury at a first location along the spinal cord, the electrical stimulation may be applied by an electrode array that is implanted epidurally on the spinal cord of the patient at a second location below the first location along the spinal cord relative to the patient's brain.

Optionally, the method may include administering one or more neuropharmaceutical agents to the patient. The neuropharmaceutical agents may include at least one of a serotonergic drug, a dopaminergic drug, a noradrenergic drug, a GABAergic drug, and glycinergic drugs. By way of non-limiting examples, the neuropharmaceutical agents may include at least one of 8-OHDPAT, Way 100.635, Quipazine, Ketanserin, SR 57227A, Ondanesetron, SB 269970, Methoxamine, Prazosin, Clonidine, Yohimbine, SKF-81297, SCH-23390, Quinpirole, Buspirone, and Eticlopride.

The electrical stimulation is defined by a set of parameter values, and activation of the selected spinal circuit may generate a quantifiable result. Optionally, the method may be repeated using electrical stimulation having different sets of parameter values to obtain quantifiable results generated by each repetition of the method. Then, a machine learning method may be executed by at least one computing device. The machine learning method builds a model of a relationship between the electrical stimulation applied to the spinal cord and the quantifiable results generated by activation of the at least one spinal circuit. A new set of parameters may be selected based on the model. By way of a non-limiting example, the machine learning method may implement a Gaussian Process Optimization.

Another exemplary embodiment is a method of enabling one or more functions selected from a group consisting of postural and/or locomotor activity, voluntary movement of leg position when not bearing weight, voluntary movement of a hand or arm, voluntary voiding of the bladder and/or bowel, return of sexual function, consisting of breathing, swallowing, or chewing, autonomic control of cardiovascular function, digestive function, body temperature control, and normalized metabolic processes, in a human subject having a neurologically derived paralysis. The method includes stimulating the spinal cord of the subject using an electrode array while subjecting the subject to physical training that exposes the subject to relevant postural proprioceptive signals, locomotor proprioceptive signals, and supraspinal signals. At least one of the stimulation and physical training modulates in real time the electrophysiological properties of spinal circuits in the subject so the spinal circuits are activated by at least one of supraspinal information and proprioceptive information derived from the region of the subject where the selected one or more functions are facilitated.

The region where the selected one or more functions are facilitated may include one or more regions of the spinal cord that control (a) lower limbs; (b) upper limbs; (c) the subject's bladder; (d) the subject's bowel; and/or (e) the subject's sexual function. The physical training may include standing, stepping, sitting down, laying down, reaching, grasping, stabilizing sitting posture, stabilizing standing posture, bearing down, trying to breath, cough, swallow, chew, blink one's eyes, turn their head, shrug their shoulders or the like.

The electrode array may include one or more electrodes stimulated in a monopolar configuration and/or one or more electrodes stimulated in a bipolar configuration. The electrode array includes a plurality of electrodes that may have an interelectrode spacing between adjacent electrodes of about 500 μm to about 1.5 mm. The electrode array may be an epidurally implanted electrode array, a transcutaneous place electrode, or combination thereof. Such an epidurally implanted electrode array may be placed over at least one of a lumbosacral portion of the spinal cord, a thoracic portion of the spinal cord, and a cervical portion of the spinal cord or brainstem.

The stimulation may include tonic stimulation and/or intermittent stimulation. The stimulation may include simultaneous or sequential stimulation of different spinal cord regions. Optionally, the stimulation pattern may be under control of the subject.

The physical training may include inducing a load bearing positional change in the region of the subject where locomotor activity is to be facilitated. The load bearing positional change in the subject may include standing, stepping, reaching, and/or grasping. The physical training may include robotically guided training.

The method may also include administering one or more neuropharmaceuticals. The neuropharmaceuticals may include at least one of a serotonergic drug, a dopaminergic drug, a noradrenergic drug, a GABAergic drug, and a glycinergic drug.

Another exemplary embodiment is a method that includes implanting an electrode array on the patient's spinal cord, positioning the patient in a training device configured to assist with physical training that is configured to induce neurological signals in the portion of the patient's body having the paralysis, and applying electrical stimulation to a portion of a spinal cord of the patient. The induced neurological signals is below the first stimulation threshold and insufficient to activate the at least one selected spinal circuit. The electrical stimulation is below the second stimulation threshold such that the at least one selected spinal circuit is at least partially activatable by the addition of at least one of (a) a second portion of the induced neurological signals, and (b) supraspinal signals. Optionally, the electrode array may be implanted on the dura of the patient's spinal cord.

Another exemplary embodiment is a system that includes a training device configured to assist with physically training of the patient, an implantable electrode array configured to be implanted on the dura of the patient's spinal cord, a stimulation generator connected to the implantable electrode array. When undertaken, the physical training induces neurological signals in the portion of the patient's body having the paralysis. The stimulation generator is configured to apply electrical stimulation to the implantable electrode array. Electrophysiological properties of at least one spinal circuit in the patient's spinal cord is modulated by the electrical stimulation and at least one of (1) a first portion of the induced neurological signals and (2) supraspinal signals such that the at least one spinal circuit is at least partially activatable by at least one of (a) the supraspinal signals and (b) a second portion of the induced neurological signals. The induced neurological signals and supraspinal signals are below the first stimulation threshold and insufficient to activate the at least one selected spinal circuit, and the electrical stimulation applied to the implantable electrode array is below the second stimulation threshold.

Another exemplary embodiment is a system that includes means for physically training the patient to induce neurological signals in the portion of the patient's body having the paralysis, and means for applying electrical stimulation to a portion of a spinal cord of the patient. Electrophysiological properties of at least one spinal circuit in the patient's spinal cord being modulated by the electrical stimulation and at least one of a first portion of the induced neurological signals and supraspinal signals such that the at least one spinal circuit is at least partially activatable by at least one of (a) the supraspinal signals and (b) a second portion of the induced neurological signals.

### BRIEF DESCRIPTION OF THE SEVERAL VIEWS OF THE DRAWINGS

FIG. 1 summarizes recent experiments in rats that were carried out to assess the effectiveness of epidural stimulation coupled with combined drug therapy in the treatment of complete spinal cord injuries. The combination of quipazine and 8-0HDPAT with simultaneous epidural stimulation at spinal sites L2 and S1 results in robust coordinated stepping as early as one week after a complete spinal cord transection. Locomotor behavior observed from a typical rat before the injury and one week after a complete mid-thoracic spinal cord transection. The amount of body weight support provided to the rat is shown in red. One week post-injury, no spontaneous stepping activity is observed. Administration of quipazine (a 5-HT₂ receptor agonist) and 8-0HDPAT (a 5-HT_{1/7} receptor agonist) results in erratic movements. Epidural stimulation simultaneously at L2 plus S1 in combination with either quipazine or 8-0HDPAT enables plantar stepping. The combination of epidural stimulation at L2 plus S1 with the administration of quipazine plus 8-0HDPAT clearly has a synergistic effect, resulting in coordinated, plantar stepping with features resembling those observed pre-lesion. Sol, soleus; TA, tibialis anterior; MTP, metatarsal-phalangeal.

FIG. 2 illustrates step training with epidural stimulation at both L2 and S1 spinal sites in combination with use of quipazine and 8-0HDAPT (5-HT agonists) prevents degradation of neuronal function and promotes improvement of the stepping ability of spinal rats transected as adults. From top to bottom: Representative stick diagrams of left and right hindlimb movements during gait swing phase, recorded 8 weeks post-injury. The successive trajectories of the left and right limb endpoint (MTP) during a 10 s stepping sequence are shown. Blue, red, and black trajectories represent stance, drag, and swing phases. The gait diagrams reconstructed from the displacement of the left and right hindlimbs during stepping are displayed conjointly with the EMG activity of left and right soleus (“Sol”) and tibialis anterior (“TA”) muscles. Compared to a rat with no rehabilitation, the rat that received step training every other day for 7 weeks shows consistent hindlimb movements, coordination between the left and right sides, and increased recruitment of both extensor and flexor leg muscles.

FIG. 3 shows a photograph of an illustrative 1st generation high density epidural stimulating array comprising 10 electrodes.

FIG. 4 shows a schematic diagram of an illustrative laminectomy procedure for placing an epidural stimulating array over the lumbosacral spinal cord.

FIGS. 5A-5D illustrate results for site-specific selective muscle activation. The extensor digitorum longus (EDL, panel A), vastus lateralis (VL, panel B), and tibialis anterior (TA, panel C) muscles were selectively activated using low-current stimulation at specific spinal sites. Preferential activation of the medial gastrocnemius (MG, panel D) muscle also was obtained, but occurred with co-activation of the VL. Data represent normalized peak-to-peak amplitudes of 10 averaged responses.

FIG. 6 shows that interelectrode distance modulates muscle recruitment. Using a smaller spacing (1500 μm, filled bars) bipolar configuration, graded muscle activation was achieved. With larger spacing (4500 μm, unfilled bars), approaching a monopolar configuration, a muscle quickly attained maximal activation at low currents. Thus, the specific goal and sensitivity requirements of a particular motor task may dictate optimal interelectrode spacing and whether a monopolar or bipolar configuration is chosen.

FIG. 7 shows a photograph of an illustrative 27 electrode rat epidural stimulation array (in a 9×3 configuration), including head-connector.

FIG. 8 shows a photograph of an illustrative 256 electrode array.

FIG. 9 illustrates a schematic of a step training robot. Illustrative components include: A) Optical encoder; B) Motor; C) Weight support; D) Manipulators; and E) Motorized treadmill.

FIGS. 10A and 10B show radiographic and clinical characteristics of an individual with motor complete, but sensory incomplete SCI. FIG. 10A: T2 weighted sagittal Magnetic Resonance Image of cervical spine at subject's injury site (C7-T1). Hyperintensity and myelomalacia noted at site of injury. FIG. 10B: AIS evaluation of the subject.

FIGS. 11A-11D illustrate localization of electrode array relative to motoneuron pools as identified with motor evoked potentials during surgical implantation. The voltage thresholds for evoked potentials of proximal muscles are lower when stimulating the more rostral electrodes. The voltage thresholds for motor evoked potentials of the distal muscles are lower when stimulating the caudal electrodes. FIG. 11A: Post-operative fluoroscopy of the thoracolumbar spine showing the location of the implanted electrode array and neurostimulator. FIG. 11B: Depiction of 16-electrode array configuration relative to spinal dorsal roots and corresponding motoneuron pools identified using EMG recorded from leg muscles. FIGS. 11C and 11D: Motor evoked potentials elicited using epidural stimulation at 2 Hz, 210 μs from 0.0 to 7 V with rostral electrodes, (5−: 6+) and caudal electrodes (10−: 9+) respectively. Muscles: IL: iliopsoas, AD: adductor magnus, VL: vastus lateralis, MH: medial hamstrings, TA: tibialis anterior, GL: gluteus maximus, SL: soleus, MG: medial gastrocnemius.

FIG. 12 illustrates lower extremity EMG activity during standing with BWST (panel A), and stepping with body weight support (“BWST”) (panel B). Three different time points over a two-year period and 170 training sessions showed no change in the EMG pattern during standing or stepping.

FIGS. 13A and 13B show EMG activity with epidural stimulation during independent standing. These data demonstrate that the output of the spinal circuitry can be sufficiently modulated by the proprioceptive input to sustain independent stepping. EMG activity increases in amplitude and becomes more constant bilaterally in most muscles with independent standing occurring at 8 V. Reducing BWS changed the EMG amplitudes and oscillatory patterns differently among muscles. EMG activity during standing with BWS and with epidural stimulation (15 Hz) of caudal lumbosacral segments (4/10/15−: 3/9+) (panel A) from 1-8V and 65% BWS and (panel B) at 8V while reducing the BWS from 45% to 5%. Muscle: rectus femoris (RF), medial hamstrings (MH), tibialis anterior (TA), and medial gastrocnemius (MG). Left (L) and right (R).

FIGS. 14A-14E illustrate lower extremity EMG activity during sitting and standing with and without epidural stimulation. There was little or no EMG activity without stimulation, but with epidural stimulation there was significant EMG activity that was modulated during the transition from sitting to standing. Panel A: EMG activity during sitting (green) and standing (yellow) with no epidural stimulation. Panel B: EMG activity during sitting (green) and standing (yellow) with 4V to 7.5 V, 15 Hz stimulation of the rostral lumbar segments (0/5/11−: 1/6/12+). Panel C: EMG activity during sitting (green) and standing (yellow) with epidural stimulation (15 Hz) of the caudal lumbosacral segments (4/10/15−: 3/9/14+). Panel D: Averaged mean amplitude (mV) of right side motor evoked responses during sitting and standing elicited from epidural stimulation (b) or rostal stimulation is represented by “▴,” (c) or caudal stimulation is represented by “♦,” and no stimulation is represented by opened circles (o). No stimulation values are only shown for sitting and standing. Panel E: Kinematic representation of transition from sitting to standing with caudal stimulation. Muscles: iliopsoas (IL), vastus lateralis (VL), medial hamstrings (MH), tibialis anterior (TA), soleus (Sol), and medial gastrocnemius (MO). Left side muscles (L), right side muscles (R).

FIGS. 15A and 15B illustrate EMG activity with epidural stimulation during independent standing. Panel A: EMG activity with epidural stimulation (8 V, 15 Hz) of the caudal lumbosacral segments (4/10/15−: 3/9/14+) during weight shifting. Body movements are depicted in the top panel as displacement of the center of gravity (CGX) lateral shifting (CGY) to the right (R)) and left (L) sides in the bottom panels. Panel B: EMG activity with epidural stimulation during independent standing. Interpulse interval depicting stimulation frequency is shown on the lower right of the top and bottom graphs. Red line indicates initiation of independent standing as subject counted backwards from 3, blue line indicates when independent standing was obtained. Muscle: iliopsoas (IL), rectus femoris (RF), medial hamstrings (MH), tibialis anterior (TA), Soleus (SOL) and medial gastrocnemius (MG). Left (L) and right (R).

FIGS. 16A-16D show lower extremity EMG activity during standing and stepping with body weight support and manual facilitation with and without epidural stimulation of caudal lumbosacral segments. The EMG patterns were modified by the intensity of stimulation and by different patterns of sensory input. EMG activity during stepping (50% BWS, 1.07 m/s) (panel A) without stimulation and (panel B) (45% BWS, 0.8 m/s) with epidural stimulation (5.5 V, 30 Hz) of caudal lumbosacral segments (4/10/15−: 3/9+). EMG activity during (panel C) standing (25% BWS) and (panels B, D) stepping (50% BWS, 1.07 m/s) with epidural stimulation (7.0 V, 30 Hz) of caudal lumbosacral segments (4/10/15−: 3/9+) (panel C). For stepping (panels B, C, and D) data were selected from 5 consecutive cycles. Muscles: vastus lateralis (VL), medial hamstrings (MH), tibialis anterior (TA), and medial gastrocnemius (MG). Left (L) and right (R) side muscles. Load is load cell reading in Newtons (N). Left (LHip) and Right (RHip) are sagittal joint angles for the hip joint. Left (LFS) and right (RFS) footswitches reflect stance phase.

FIGS. 17A-17E show lower extremity EMG activity during voluntary control in a supine position with and without stimulation. The black bar indicates the command to generate flexion and move the left leg up (FIG. 17A), left ankle dorsiflexion (FIG. 17B), and left toe extension (FIG. 17C), and the white bar indicates the command to relax the leg. Left and right sides are shown to emphasize the isolated control of the left side following the command. The right and left intercostals (IC) are activated during the voluntary attempt of the leg, as the subject inhales as he attempts to perform the movement. Muscles: soleus (SOL), extensor digitorum longus (EDL), extensor hallucis longus (EHL), tibialis anterior (TA), peroneus longus (PL), vastus lateralis (VL), medial hamstrings (MH), adductor magnus (AD), gluteus maximus (GL), iliopsoas (IL), erector spinae (ES), rectus abdominus (AB), intercostals (IC). Sagittal joint angles for the toe (1st metatarsal relative to foot), ankle, knee, and hip joints. FIG. 17D: Stick figures were generated from the kinematics during the up and down commands for both trials with and without epidural stimulation. FIG. 17E: Relationship between onset (solid)/offset (open) of EMG burst for TA muscle and command up/down. Three trials were performed for the toe and leg voluntary movements and two trials for the ankle. All commands were given to move the left leg. The dotted line represents the line of identity (x=y).

FIG. 18A shows a 3D view of epidural spinal electrode (with 2 of 27 electrodes activated) placed in the epidural space of a simulated spinal cord.

FIG. 18B shows isopotential contours of electrical field (in slice through center of bipolarly activated electrodes). Model compartments include gray matter, white matter, CSF, epidural fat, and surrounding body tissue.

FIG. 19 (top) shows instantaneous regret (a measure of machine learning error) vs. learning iteration (labeled as “query number”) for Gaussian Process Optimization of array stimulation parameters in the simulated spinal cord of FIGS. 18A and 18B. The “bursts” of poor performance corresponds to excursions of the learning algorithm to regions of parameter space that are previously unexplored, but which are found to have poor performance. FIG. 19 (bottom) shows the average cumulative regret vs. learning iteration. The average cumulative regret is a smoothed version of the regret performance function which better shows the algorithm's overall progress in selecting optimal stimulation parameters.

FIGS. 20A-20F illustrate representative EMG and evoked potentials with and without eEmc. Representative raw EMG and evoked potentials from the soleus and tibialis anterior (TA) muscles without eEmc from one spinal rat during (A) sitting, (B) attempted bipedal standing, and with eEmc (1.5 V, 40 Hz between L2 and S1) during (C) sitting, (D) bipedal standing, and (E) quadrupedal (Quad) stepping-like movement during the 6-hr recording period in its home cage. (F) Representative EMG and evoked potential from the soleus and TA from the same rat during body weight supported bipedal treadmill stepping facilitated by eEmc (2.0 V, 40 Hz between L2 and S1). The start of each trace with eEmc is synchronized with the initiation of the eEmc pulse. Each trace is 25 msec, i.e., the time between successive eEmc pulses. The arrow placed on the EMG signals denotes the time of the initial 25 msec scan.

FIG. 21 illustrates total activity time with and without eEmc. Mean (±SEM, n=4) duration of spontaneous cage activity during the 6-hr recording period with and without eEmc. *, significantly different from without eEmc at P<0.05.

FIG. 22 Integrated EMG during spontaneous cage activity and treadmill locomotion. (A) Integrated EMG during body weight supported treadmill stepping at 13.5 cm/sec for 1 min, (B) integrated EMG per min for the TA and soleus (sol) during the 6-hr recording period in the cage, and (C) sum of the integrated EMG during the 6-hr recording period in the cage for the TA and soleus muscles without and with eEmc. Values are mean±SEM for 4 rats. *, significantly different from without eEmc at P<0.05.

FIG. 23 illustrates frequency distribution of integrated EMG. Frequency distribution of the mean (±SEM, n=4 rats) integrated EMG amplitudes for the TA and soleus with and without eEmc during the 6-hr recording period in the cage expressed in one-min bins. *, significantly different from the corresponding bin without eEmc at P<0.05.

FIG. 24 illustrates average integrated EMG with and without eEmc. Mean (±SEM) frequency of occurrence of different ranges of integrated EMG amplitudes with and without eEmc during the 6-hr recording period of cage activity expressed in one-min bins.

FIGS. 25A and 25B illustrate JPD plots from a single animal throughout the 6 hours with and without eEmc. (A) Joint probability distribution plots showing the relationship between the soleus and TA activity expressed in 10-min bins during the 6-hr recording period for a representative spinal rat. The 6-hr recording occurred during the dark period (8:00 μm to 2:00 am), i.e., the active period of the rats. (B) The incidence of occurrence of different joint probability distributions for 10 min of activity without (I) and with (II) eEmc. The asterisks in (A) identify the two bins being compared in (B), without eEmc (I) and with eEmc (II). Note the lack of consistent alternating flexor-extensor activation without compared to with eEmc.

### DETAILED DESCRIPTION

Described herein are systems and methods for providing sub-threshold spinal stimulation. This stimulation can be delivered using an implanted or epidural stimulator using one or more electrodes or electrode arrays. This stimulation can produce one or more spontaneous voluntary movement.

In some embodiments, electrical enabling motor control (eEmc) combined with spontaneous activity may increase the frequency and level of activation of the locomotor circuits in paralyzed individuals. In some embodiments, this increase can result in spontaneous voluntary movement.

Spontaneous voluntary (or supraspinal) movement can be of a toe, ankle, leg, knee, finger, elbow, shoulder, hand, hip, chest extension or contraction, trunk flexion or rotation, neck flexion or extension, movement of erect penis, or a combination thereof.

In some embodiments, in the presence of eEmc, an individual or mammal can become about 2 times, about 3 times, about 4 times, about 5 times, about 6 times, about 7 times, about 8 times, about 9 times, about 10 times, about 20 times, about 50 times, at least about 2 times, at least about 5 times, or at least about 10 times more active than without eEmc delivery.

Unless defined otherwise, technical and scientific terms used herein have the same meaning as commonly understood by one of ordinary skill in the art to which this invention belongs.

In some embodiments, eEmc can be delivered below the level of a spinal lesion and thereby enhance the amount of spontaneous voluntary movement or activity. In some embodiments, this enhanced amount of spontaneous voluntary movement can result in more robust stepping-like and partial weight bearing standing activities.

In some embodiments, in addition to eEmc being administered with training and/or therapeutic agents, the environment of the individual can aid in bringing about spontaneous voluntary movements. For example, training within one's own home or another positive environment can assist in treatment.

In some embodiments, an enhanced amount of spontaneous voluntary movement or activity in the sensorimotor circuits that can be achieved that can facilitate standing and stepping in individuals suffering from spinal injuries using chronic sub-threshold eEmc.

The term “motor complete” when used with respect to a spinal cord injury indicates that there is no motor function below the lesion, (e.g., no movement can be voluntarily induced in muscles innervated by spinal segments below the spinal lesion (e.g., as described below in Example 1).

The term “bipolar stimulation” refers to stimulation between two closely spaced electrodes.

The term “monopolar stimulation” refers to stimulation between a local electrode and a common distant return electrode.

The term “autonomic function” refers to functions controlled by the autonomic nervous system that are controlled largely below the level of consciousness, and typically involve visceral functions. Illustrative autonomic functions include, but are not limited to control of bowel, bladder, and body temperature.

The term “sexual function” refers to the ability to sustain a penile erection, have an orgasm (male or female), generate viable sperm, and/or undergo an observable physiological change associated with sexual arousal.

The term “cognitive function” refers to awareness of one's surrounding environment and the ability to function effectively, behaviorally, and mentally in a given environment.

In various embodiments, methods, devices, and optional pharmacological agents are provided to facilitate movement in a mammalian subject (e.g., a human) having spinal cord injury, brain injury, or other neurological disease or injury. In certain embodiments, the methods involve stimulating the spinal cord of the subject using an electrode array where the stimulation modulates the electrophysiological properties of selected spinal circuits in the subject so they can be activated by proprioceptive derived information and/or input from supraspinal. In various embodiments, the stimulation is typically accompanied by physical training (e.g., movement) of the region where the sensory-motor circuits of the spinal cord are located.

In particular illustrative embodiments, the devices, optional pharmacological agents, and methods described herein stimulate the spinal cord with, e.g., electrode arrays, that modulate the proprioceptive and supraspinal information which controls the lower limbs during standing and/or stepping and/or the upper limbs during reaching and/or grasping conditions. It is the sensory information that guides the activation of the muscles in a coordinated manner and in a manner that accommodates the external conditions, e.g., the amount of loading, speed, and direction of stepping or whether the load is equally dispersed on the two lower limbs, indicating a standing event, alternating loading indicating stepping, or sensing postural adjustments signifying the intent to reach and grasp.

Unlike approaches that involve specific stimulation of motor neurons to directly induce a movement, the methods described herein enable the spinal circuitry to control the movements. More specifically, the devices, optional pharmacological agents, and methods described herein exploit the spinal circuitry and its ability to interpret proprioceptive information and to respond to that proprioceptive information in a functional way. In various embodiments, this is in contrast to other approaches where the actual movement is induced/controlled by direct stimulation (e.g., of particular motor neurons).

In one illustrative embodiment, the subject is fitted with one or more implantable electrode arrays that afford selective stimulation and control capability to select sites, mode(s), and intensity of stimulation via electrodes placed epidurally over, for example, the lumbosacral spinal cord and/or cervical spinal cord to facilitate movement of the arms and/or legs of individuals with a severely debilitating neuromotor disorder.

The subject receives the implant (a standard procedure when used for pain alleviation), and typically about two weeks post implant, the subject is tested to identify the most effective subject specific stimulation paradigms for facilitation of movement (e.g., stepping and standing and/or arm and/or hand movement). Using these stimulation paradigms, the subject practices standing and stepping and/or reaching or grabbing in an interactive rehabilitation program while being subject to spinal stimulation.

Depending on the site/type of injury and the locomotor activity it is desired to facilitate, particular spinal stimulation protocols include, but are not limited to specific stimulation sites along the lumbosacral and/or cervical spinal cord; specific combinations of stimulation sites along the lumbosacral and/or cervical spinal cord; specific stimulation amplitudes; specific stimulation polarities (e.g., monopolar and bipolar stimulation modalities); specific stimulation frequencies; and/or specific stimulation pulse widths.

In various embodiments, the system is designed so that the patient can use and control it in the home environment.

In various embodiments, the approach is not to electrically induce a walking pattern or standing pattern of activation, but to enable/facilitate it so that when the subject manipulates their body position, the spinal cord can receive proprioceptive information from the legs (or arms) that can be readily recognized by the spinal circuitry. Then, the spinal cord knows whether to step or to stand or to do nothing. In other words, this enables the subject to begin stepping or to stand or to reach and grasp when they choose after the stimulation pattern has been initiated.

Moreover, as demonstrated in Example 1 (described below), the methods and devices described herein are effective in a spinal cord injured subject that is clinically classified as motor complete; that is, there is no motor function below the lesion. In various embodiments, the specific combination of electrodes activated/stimulated within an array and/or the desired stimulation of any one or more electrodes and/or the stimulation amplitude (strength) can be varied in real time, e.g., by the subject. Closed loop control can be embedded in the process by engaging the spinal circuitry as a source of feedback and feedforward processing of proprioceptive input and by voluntarily imposing fine tuning modulation in stimulation parameters based on visual, and/or kinetic, and/or kinematic input from selected body segments.

In various embodiments, the devices, optional pharmacological agents, and methods are designed so that a subject with no voluntary movement capacity can execute effective standing and/or stepping and/or reaching and/or grasping. In addition, the approach described herein can play an important role in facilitating recovery of individuals with severe although not complete injuries.

The approach described herein can provide some basic postural, locomotor and reaching and grasping patterns on their own. However, they are also likely to be a building block for future recovery strategies. Based on certain successes in animals and some preliminary human studies (see below), it appears that a strategy combining effective epidural stimulation of the appropriate spinal circuits with physical rehabilitation and pharmacological intervention can provide practical therapies for complete SCI human patients. There is sufficient evidence from our work that such an approach should be enough to enable weight bearing standing, stepping and/or reaching or grasping. Such capability can give complete SCI patients the ability to participate in exercise, which is known to be highly beneficial for their physical and mental health. We also expect our method should enable movement with the aid of assistive walkers. While far from complete recovery of all movements, even simple standing and short duration walking would increase these patients' autonomy and quality of life. The stimulating array technology described herein (e.g., epidural stimulating arrays) paves the way for a direct brain-to-spinal cord interface that could enable more lengthy and finer control of movements.

While the methods and devices described herein are discussed with reference to complete spinal injury, it will be recognized that they can apply to subjects with partial spinal injury, subjects with brain injuries (e.g., ischemia, traumatic brain injury, stroke, and the like), and/or subjects with neurodegenerative diseases (e.g., Parkinson's disease, Alzheimer's disease, Huntington's disease, amyotrophic lateral sclerosis (ALS), primary lateral sclerosis (PLS), cerebral palsy, and the like).

In various embodiments, the methods combine the use of epidural stimulating arrays with physical training (e.g., rigorously monitored (robotic) physical training), optionally in combination with pharmacological techniques. The methods enable the spinal cord circuitry to utilize sensory input as well as newly established functional connections from the brain to circuits below the spinal lesion as a source of control signals. The approach is thus designed to enable and facilitate the natural sensory input as well as supraspinal connections to the spinal cord in order to control movements, rather than induce the spinal cord to directly induce the movement. That is, we facilitate and enhance the intrinsic neural control mechanisms of the spinal cord that exist post-SCI, rather than replace or ignore them.

### Processing of Sensory Input by the Lumbosacral Spinal Cord: Using Afferents as a Source of Control

In various embodiments the methods and devices described herein exploit spinal control of locomotor activity. For example, the human spinal cord can receive sensory input associated with a movement such as stepping, and this sensory information can be used to modulate the motor output to accommodate the appropriate speed of stepping and level of load that is imposed on lower limbs. Moreover, we have demonstrated that the human lumbosacral spinal cord has central-pattern-generation-like properties. Thus, oscillations of the lower limbs can be induced simply by vibrating the vastus lateralis muscle of the lower limb, by epidural stimulation, and by stretching the hip. The methods described herein exploit the fact that the human spinal cord, in complete or incomplete SCI subjects, can receive and interpret proprioceptive and somatosensory information that can be used to control the patterns of neuromuscular activity among the motor pools necessary to generate particular movements, e.g., standing, stepping, reaching, grasping, and the like. The methods described herein facilitate and adapt the operation of the existing spinal circuitry that generates, for example, cyclic step-like movements via a combined approach of epidural stimulation, physical training, and, optionally, pharmacology.

### Facilitating Stepping and Standing in Humans Following a Clinically Complete Lesion

Locomotion in mammals is attributed to intrinsic oscillating spinal neural networks capable of central pattern generation interacting with sensory information (Edgerton et al., J. American Paraplegia Soc, 14(4) (1991), 150-157; Forssberg, J. Neurophysiol, 42(4): 936-953 (1979); Grillner and Wallen, Annu. Rev. Neurosci., 8: 233-261 (1985); Grillner and Zangger, Exp Brain Res, 34(2): 241-261 (1979)). These networks play critical roles in generating the timing of the complex postural and rhythmic motor patterns executed by motor neurons.

As indicated above, the methods described herein can involve stimulation of one or more regions of the spinal cord in combination with locomotory activities. It was our discovery that spinal stimulation in combination with locomotor activity results in the modulation of the electrophysiological properties of spinal circuits in the subject so they are activated by proprioceptive information derived from the region of the subject where locomotor activity is to be facilitated. Further, we also determined that spinal stimulation in combination with pharmacological agents and locomotor activity results in the modulation of the electrophysiological properties of spinal circuits in the subject so they are activated by proprioceptive information derived from the region of the subject where locomotor activity is to be facilitated.

Locomotor activity of the region of interest can be accomplished by any of a number of methods known, for example, to physical therapists. By way of illustration, individuals after severe SCI can generate standing and stepping patterns when provided with body weight support on a treadmill and manual assistance. During both stand and step training of human subjects with SCI, the subjects can be placed on a treadmill in an upright position and suspended in a harness at the maximum load at which knee buckling and trunk collapse can be avoided. Trainers positioned, for example, behind the subject and at each leg assist as needed in maintaining proper limb kinematics and kinetics appropriate for each specific task. During bilateral standing, both legs can be loaded simultaneously and extension can be the predominant muscular activation pattern, although co-activation of flexors can also occur. Additionally, or alternatively, during stepping the legs are loaded in an alternating pattern and extensor and flexor activation patterns within each limb also alternated as the legs moved from stance through swing. Afferent input related to loading and stepping rate can influence these patterns, and training has been shown to improve these patterns and function in clinically complete SCI subjects.

### Epidural Stimulation of the Lumbosacral Spinal Cord

As indicated above, without being bound by a particular theory, it is believed that epidural stimulation, e.g., over the lumbosacral spinal cord in combination with physical training can facilitate recovery of stepping and standing in human subjects following a complete SCI.

Spinal cord electrical stimulation has been successfully used in humans for suppression of pain and spasticity (see, e.g., Johnson and Burchiel, Neurosurgery, 55(1): 135-141 (2004); discussion 141-142; Shealy et al., Anesth Analg, 46(4): 489-491 (1967); Campos et al., Appl. Neurophysiol. 50(1-6): 453-454 (1987); Dimitrijevic and Sherwood, Neurology, 30 (7 Pt 2): 19-27 (1980); Barolat Arch. Med. Res., 31(3): 258-262 (2000); Barolat, J. Am. Paraplegia Soc., 11(1): 9-13 (1988); Richardson et al., Neurosurgery, 5(3): 344-348). Recent efforts to optimize electrode design and stimulation parameters have led to a number of research studies focusing on the benefits of epidural spinal cord stimulation. We have demonstrated that the location of the electrode array and its stimulation parameters are important in defining the motor response. Use of high density electrode arrays, as described herein, facilitates selection or alteration of particular stimulation sites as well as the application of a wide variety of stimulation parameters.

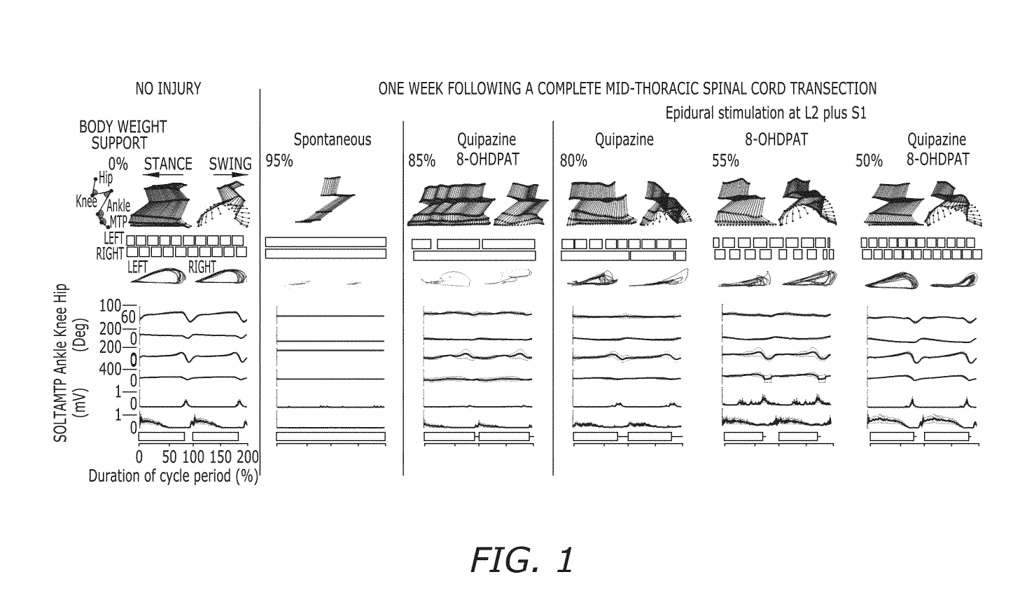

FIG. 1 summarizes experiments in rats that were carried out to assess the effectiveness of epidural stimulation coupled with combined drug therapy in acute treatment of complete spinal cord injury. These experiments also show that pharmacological intervention provides some recovery of stepping function, but that epidural stimulation coupled with drug therapy recovers significant amounts of stepping ability even one week after a complete spinal transaction.

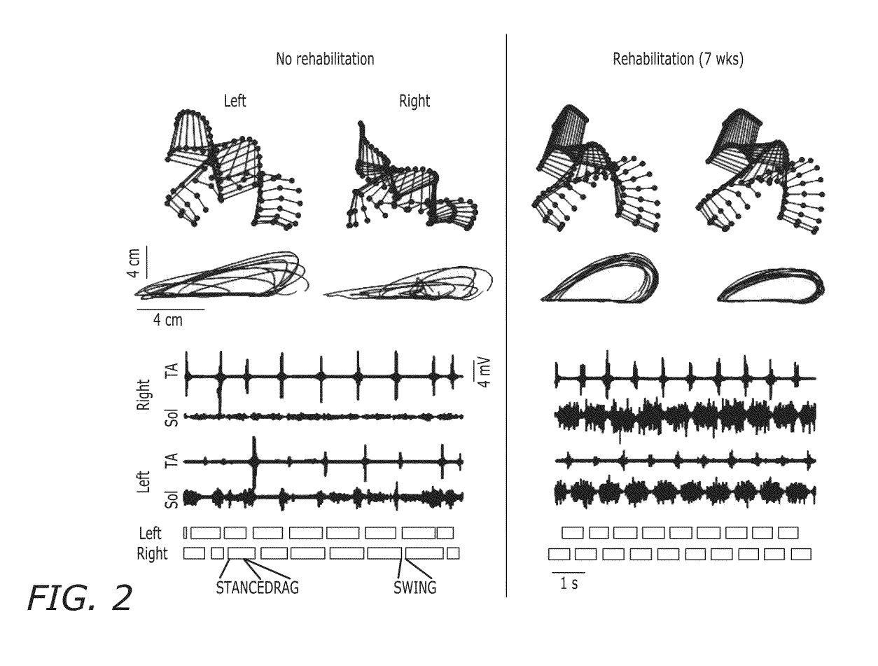

FIG. 2 compares two adult rats with complete spinal cord transections at the end of a 7 week period during which both animals were given both drug therapy as well as epidural stimulation (using conventional rod-electrodes at two spinal sites). The animal which was also given robotically guided physical therapy showed significant improvement over the animal which did not receive physical training. These results provide support for our assertion that a strategy that combines physical therapy with epidural stimulation and, optional, pharmacological modulation of the post-SCI spinal circuits can facilitate standing and stepping recovery in humans.

### MicroFabricated High-Density Epidural Stimulating Arrays

In various embodiments, the epidural electrical stimulation is administered via a high density epidural stimulating array. In certain embodiments, the high density electrode arrays use microfabrication technology to place numerous electrodes in an array configuration on a flexible substrate. One suitable epidural array fabrication method was first developed for retinal stimulating arrays (see, e.g., Maynard, Annu. Rev. Biomed. Eng., 3: 145-168 (2001); Weiland and Humayun, IEEE Eng. Med. Biol. Mag., 24(5): 14-21 (2005)), and U.S. Patent Publications 2006/0003090 and 2007/0142878 which are incorporated herein by reference for all purposes (e.g., the devices and fabrication methods disclosed therein). In various embodiments the stimulating arrays comprise one or more biocompatible metals (e.g., gold, platinum, chromium, titanium, iridium, tungsten, and/or oxides and/or alloys thereof) disposed on a flexible material (e.g., parylene A, parylene C, parylene AM, parylene F, parylene N, parylene D, or other flexible substrate materials). Parylene has the lowest water permeability of available microfabrication polymers, is deposited in a uniquely conformal and uniform manner, has previously been classified by the FDA as a United States Pharmacopeia (USP) Class VI biocompatible material (enabling its use in chronic implants) (Wolgemuth, Medical Device and Diagnostic Industry, 22(8): 42-49 (2000)), and has flexibility characteristics (Young's modulus ˜4 GPa (Rodger and Tai, IEEE Eng. Med. Biology, 24(5): 52-57 (2005))), lying in between those of PDMS (often considered too flexible) and most polyimides (often considered too stiff). Finally, the tear resistance and elongation at break of parylene are both large, minimizing damage to electrode arrays under surgical manipulation (Rodger et al., Sensors and Actuators B-Chemical, 117(1): 107-114 (2006)).

The electrode array may be implanted using any of a number of methods (e.g., a laminectomy procedure) well known to those of skill in the art.

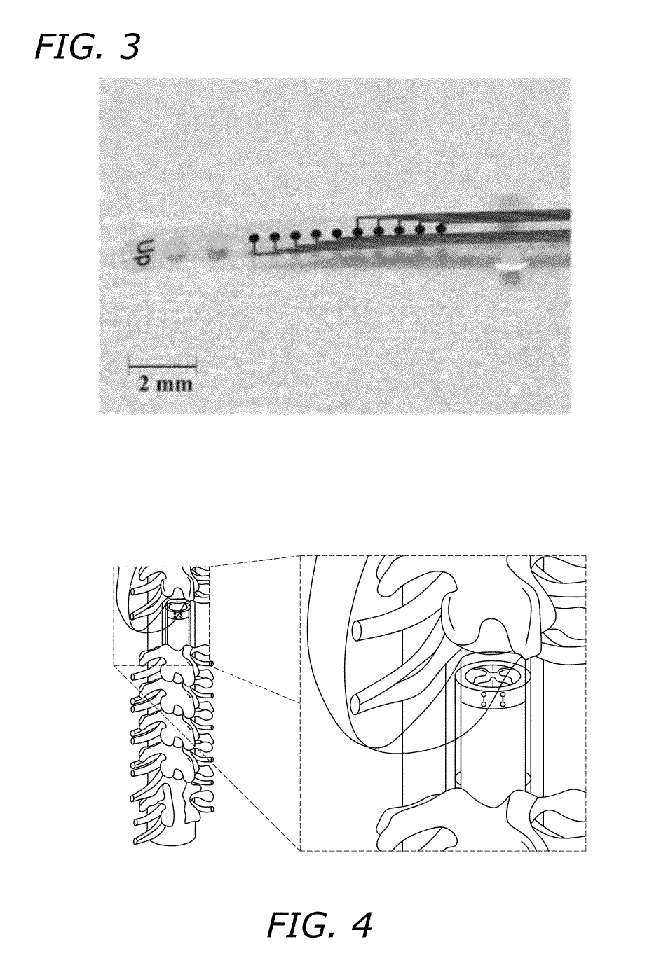

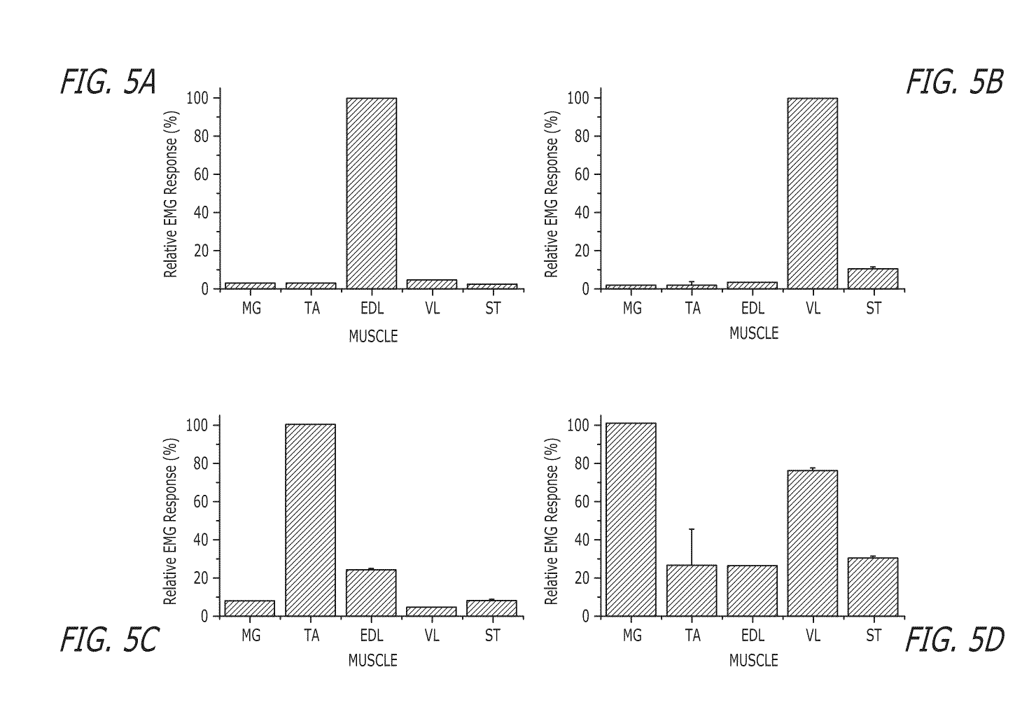

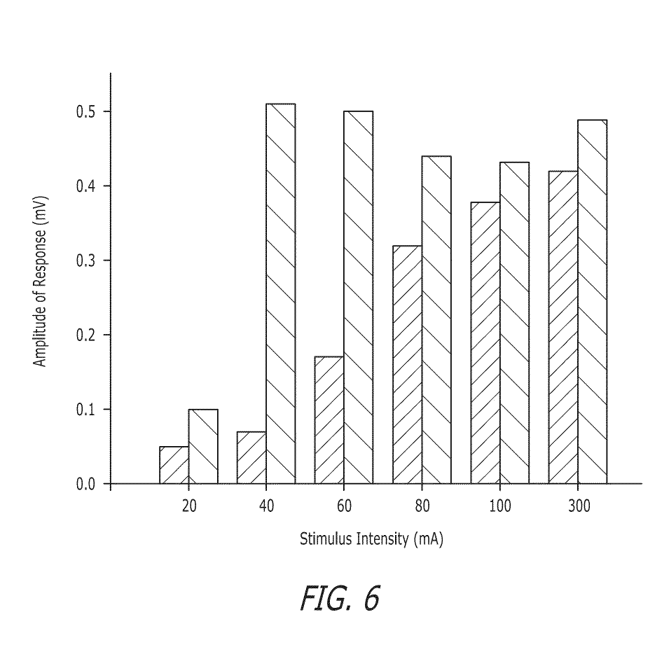

FIG. 3 shows a first prototype microelectrode array, scaled for mice, in which ten 250 micron diameter platinum electrodes are microfabricated onto a 2 mm wide Parylene backing. The electrodes are dorsally implanted using a laminectomy over the lumbosacral spinal cord, with one electrode placed over each intravertebral segment. In chronic implantation studies (using rat, mice, and pig animal models) of up to 6 months, we have shown high biocompatibility of these arrays with mammalian tissue. Implantation of an array into a human subject is described in Example 1.

Of course, other microarray embodiments are contemplated. In certain embodiments, the number of electrodes formed on an electrode array can vary from one electrode to about 100,000 electrodes or more. In certain embodiments, the electrode microarray comprises at least 10, at least 15, at least 20, at least 25, at least 50, at least 100, at least 250, at least 500, or at least 1000 electrodes. In various embodiments the interelectrode spacing of adjacent electrodes in the electrode array varies from about 100 μm or about 500 μm, or about 1000 μm or about 1500 μm to about 2000 μm, or about 3000 μm, or about 4000 μm, or about 4500 μm, or about 5000 μm. In various embodiments, interelectrode spaking ranges from about 100 μm, about 150 μm, about 200 μm, or about 250 μm up to about 1,000 μm, about 2000 μm, about 3000 μm, or about 4,000 μm. In various illustrative embodiments, individual electrode diameters (or width) range from about 50 μm, 100 μm, 150 μm, 200 μm, or 250 μm up to about 500 μm, about 1000 μm, about 1500 μm, or about 2000 μm.

The electrode array can be formed in any geometric shape such as a square or circular shape; typically the size of the array will be on the order of about 0.1 mm to about 2 cm, square or in diameter, depending in part on the number of electrodes in the array. In various embodiments, the length of an electrode array ranges from about 0.01 mmm, or 0.1 mm up to about 10 cm or greater.

In various embodiments, the arrays are operably linked to control circuitry that permits selection of electrode(s) to activate/stimulate and/or that controls frequency, and/or pulse width, and/or amplitude of stimulation. In various embodiments, the electrode selection, frequency, amplitude, and pulse width are independently selectable, e.g., at different times, different electrodes can be selected. At any time, different electrodes can provide different stimulation frequencies and/or amplitudes. In various embodiments, different electrodes or all electrodes can be operated in a monopolar mode and/or a bipolar mode, using constant current or constant voltage delivery of the stimulation.

In certain embodiments, the electrodes can also be provided with implantable control circuitry and/or an implantable power source. In various embodiments, the implantable control circuitry can be programmed/reprogrammed by use of an external device (e.g., using a handheld device that communicates with the control circuitry through the skin). The programming can be repeated as often as necessary.

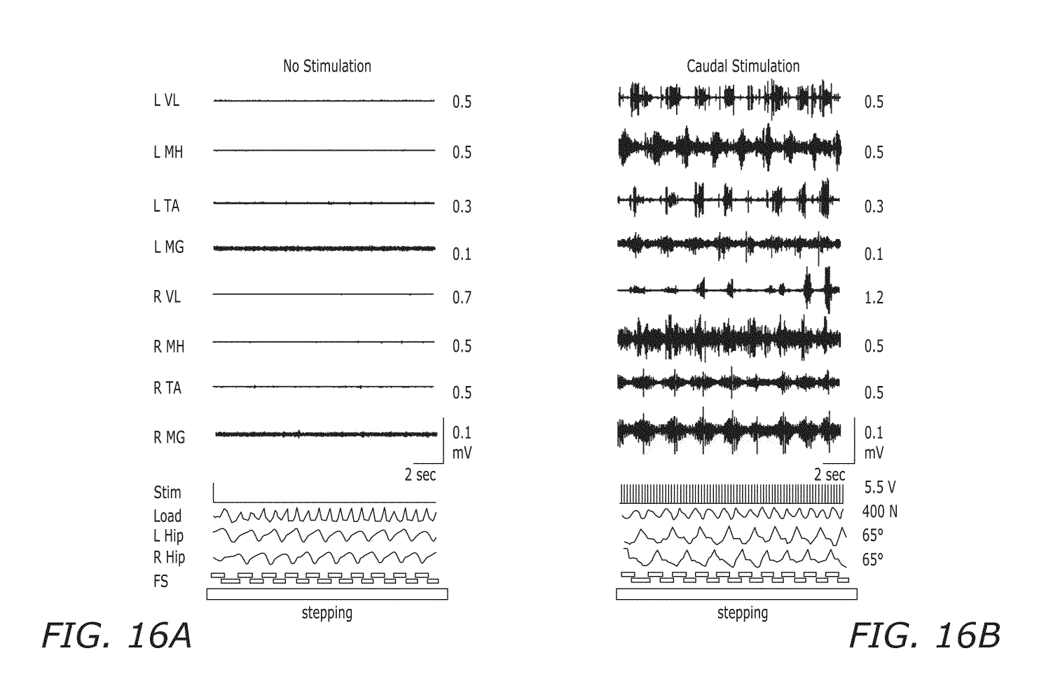

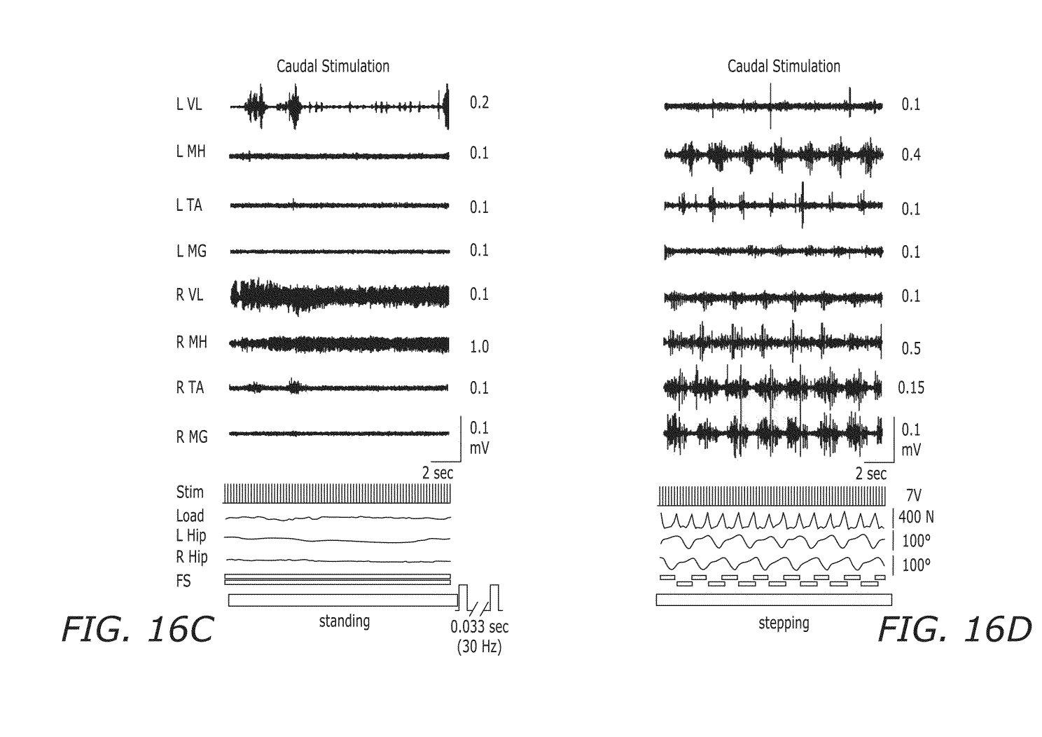

FIG. 16 shows EMG responses from different muscle groups to different types of stimulation (monopolor and bipolar) at different spinal sites. These data show that our strategy of spatially selective epidural stimulation of different portions of the lumbosacral spinal cord can focally excite and coordinate the muscle groups that are involved in locomotion.

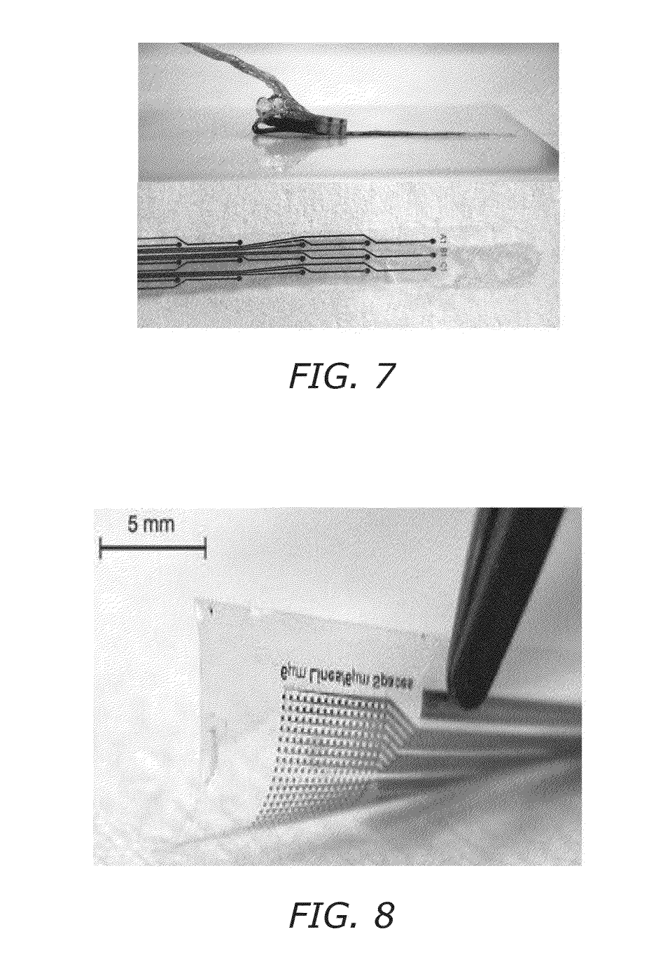

We have also developed and tested in rats more complex twenty-seven electrode arrays, which are arranged in a 9×3 pattern so that there are 3 electrodes (mid-line, left, and right) at each of 9 intravertebral segments (FIG. 7). These arrays have been tested for up to 6 weeks in vivo, showing biocompatibility as well as stepping capability that betters the previous results we have obtained with conventional electrodes. FIG. 8 shows a 256 electrode array that was fabricated to demonstrate the potential for multi-layer fabrication technology to build an array of hundreds of electrodes.

Embodiments of the electrode arrays described herein may be constructed to offer numerous advantages. For example, flexible parylene electrode arrays are mechanically stable. Their flexibility allows them to conform to the contours of the spinal cord, forming a thin layer (e.g., 10 μm thick) that adheres to the cord. This close fit facilitates connective tissue encapsulation, which also enhances fixation.

The arrays may also offer spatially selective stimulation. Early studies of stimulation protocols to facilitate locomotion in SCI animals delivered stimuli to a single spinal cord region as the ideal stimulation site was hypothesized to be fixed and species-specific. Researchers identified “optimal” stimulation sites for cats (Gerasimenko et al., Neurosci. Behav. Physiol., 33(3): 247-254 (2003)) and for rats (Gerasimenko et al., J Neurosci. Meth., 157(2): 253-263 (2006)) at a single time point after injury. However, the optimal stimulation site may not be constant. Rat studies showed that while stimulation at the L2 spinal level facilitated the best stepping soon after a complete transection, S1 stimulation produced more effective stepping several weeks later (Id.). Similarly, clinical data from patients receiving SCS for the treatment of lower back pain indicates that continued pain suppression often requires adjustment of the electrode position (Carter, Anaesth. Intensive Care, 32(1): 11-21 (2004)). These data support the hypothesis that the optimal stimulation pattern is not fixed. After a traumatic injury, the spinal cord is continuously modified by the progression of secondary damage, as well as the post-injury therapies. Our arrays' high electrode density enables ongoing identification of the optimal stimulation patterns. Our arrays' high-density allows adjustment of the stimulating pattern to account for migration, or for initial surgical misalignment.

The electrode arrays described herein also facilitate the use of advanced stimulation paradigms. Given the complex chain of reflexes involved, for example, in stepping, we believe that more sophisticated spatiotemporal stimulation patterns, involving either simultaneous or sequential stimulation of different spinal cord regions, may facilitate improved posture and locomotion and reaching and grasping compared with simple patterns. The high electrode densities allow us to test advanced stimulation paradigms that have previously been infeasible to study.

In addition, the electrode arrays provide for a lower charge injection amplitude and lower power consumption. The close positioning to the spinal cord possible with electrode arrays described herein minimizes the required levels of charge injection and power consumption. Since long-term tissue damage caused by electrical stimulation is proportional to injected charge, our conformal arrays allow longer sustained bouts of stimulation. This is desirable for long-term stimulation therapy and for battery-powered implants.

The electrode arrays described herein facilitate the measurement and evaluation of evoked potentials. Our electrode arrays can record field potentials from the dorsum (or other regions) of the spinal cord. Spinal somatosensory evoked potentials (SSEPs) measured from different levels of the spinal cord can be used to assess the state of the spinal cord and, potentially, to identify and classify the nature of a spinal injury. SSEPs are typically composed of a series of responses. With an array, response latency, amplitude, and conduction velocity can be simultaneously gathered from positions throughout the lumbosacral spinal cord. Examining the SSEPs for different injury types facilitates the generation of an injury-specific atlas of spinal potentials. SSEPs can be used as a measure of recovery and to evaluate the potential effectiveness of different treatment paradigms that might be applied. Monitoring SSEPs at different time points after the start of a treatment provides insight into the synaptic mechanisms that are involved in reacquiring locomotor function, and also serve as a diagnostic of how and if a particular strategy is aiding recovery. For example, recent data collected in our lab suggests that the return of polysynaptic spinal responses may be correlated with regaining the ability to step.

### Use of Machine Learning to Select Optimal Electrode Array Stimulation Parameters

High density epidural stimulating electrode arrays can provide patient-customized stimuli, compensate for errors in surgical placement of the array, and adapt the stimuli over time to spinal plasticity (changes in spinal cord function and connectivity). However, with this flexibility comes the burden of finding suitable stimuli parameters (e.g., the pattern of electrode array stimulating voltage amplitudes, stimulating currents, stimulating frequencies, and stimulating waveform shapes) within the vast space of possible electrode array operating patterns. It is not practical to exhaustively test all possible parameters within this huge space to find optimal parameter combinations. Such a process would consume a large amount of clinical resources. A machine learning algorithm can employed to more efficiently search for effective parameter combinations. Over time, a machine learning algorithm can also be used to continually, occasionally, and/or periodically adapt the stimulation operating parameters as needed.

A machine learning algorithm that seeks to optimize the stimuli parameters desirably alternates between exploration (searching the parameter space and building a regression model that relates stimulus and motor response) and exploitation (optimizing the stimuli patterns based on the current regression model). Many machine learning algorithms incorporate exploration and exploitation phases, and any learning algorithm that incorporates these two phases can be employed as a procedure to select (e.g., optimize) the electrode array stimulating parameters over time.

One particular embodiment relies upon Gaussian Process Optimization (GPO) (Rasmussen, Gaussian Processes for Machine Learning, MIT Press (2006)), an active learning method whose update rule explores and exploits the space of possible stimulus parameters while constructing an online regression model of the underlying mapping from stimuli to motor performance (e.g., stepping, standing, or arm reaching). Gaussian Process Regression (GPR), the regression modeling technique at the core of GPO, is well suited to online use because it requires fairly minimal computation to incorporate each new data point, rather than the extensive recomputation of many other machine learning regression of models lying within a restricted set, rather than from a single model, allowing it to avoid the over-fitting difficulties inherent in many parametric regression and machine learning methods.

GPR is formulated around a kernel function, k(,), that can incorporate prior knowledge about the local shape of the performance function (obtained from experience and data derived in previous epidural stimulation studies), to extend inference from previously explored stimulus patterns to new untested stimuli. Given a function that measures performance (e.g., stepping, standing, or reaching), GPO is based on two key formulae and the selection of an appropriate kernel function. The core GPO equation describes the predicted mean μₜ(x*) and σₜ²(x*) of the performance function (over the space of possible stimuli), at candidate stimuli x*, on the basis of past measurements (tests of stimuli values X={x₁, x₂, . . . } which returned noisy performance values Yₜ={y₁, y₂, . . . })

μₜ(x*)=k(x*,X)[Kₜ(X,X)+σₙ²I]⁻¹Yₜ;

σₜ²(x*)=k(x*,x*)−k(x*,X)[Kₜ(X,X)+σₙ²I]⁻¹k(X,x*)

where Kₜ is the noiseless covariance matrix of past data, and σₙ² is the estimated noise covariance of the data that is used in the performance evaluation. To balance exploration of regions of the stimuli space where little is known about expected performance with exploitation of regions where we expect good performance, GPO uses an upper confidence bound update rule (Srinivas and Krause, Gaussian Process Optimization in the bandit setting: No Regret and Experimental Design, Proc. Conf. on Machine Learning, Haifa Israel (2010)).

xₜ₊₁=argmax_{x13X*}[μₜ(x)+βₜσₜ(x)].

When the parameter βₜ increase with time, and if the performance function is a Gaussian process or has a low Reproducing Kernel Hilbert Space norm relative to a Gaussian process, GPO converges with high probability to the optimal action, given sufficient time.

The definition of a performance function that characterizes human motor behavior (e.g. standing or stepping behavior) typically depends upon two factors: (1) what kinds of motor performance data is available (e.g., video-based motion capture data, foot pressure distributions, accelerometers, electromyographic (EMG) measurements, etc.); and (2) the ability to quantify motor performance. While more sensory data is preferable, a machine learning approach to parameter optimization can employ various types of sensory data related to motor performance. It should be noted that even experts have great difficulty determining stepping or standing quality from such data without also looking at video or the actual subject as they undertake a motor task. However, given a sufficient number of training examples from past experiments and human grading of the standing or stepping in those experiments, a set of features that characterize performance (with respect to the given set of available sensors) can be learned and then used to construct a reasonable performance model that captures expert knowledge and that uses the available measurement data.

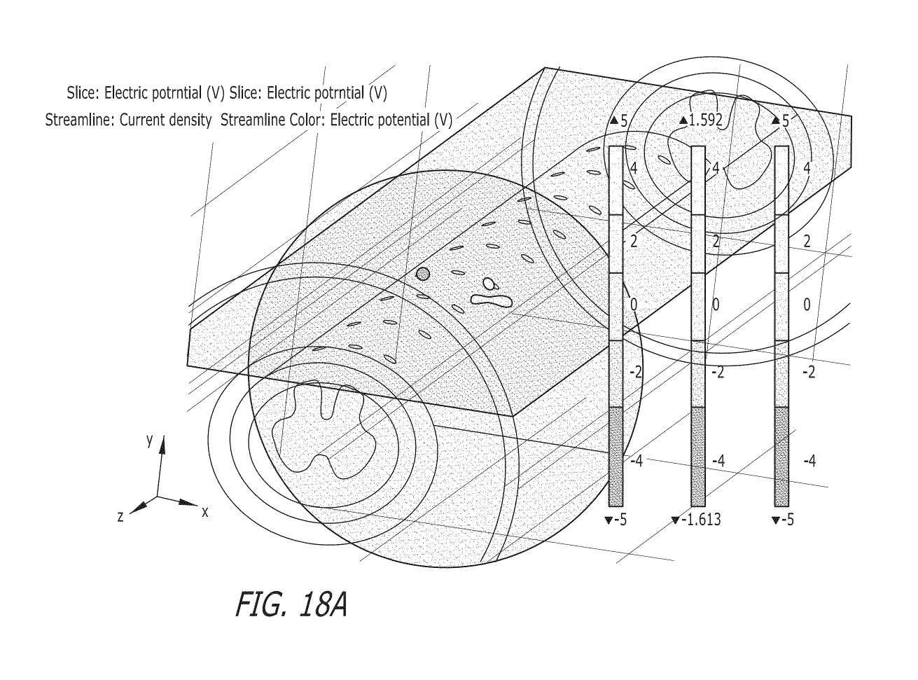

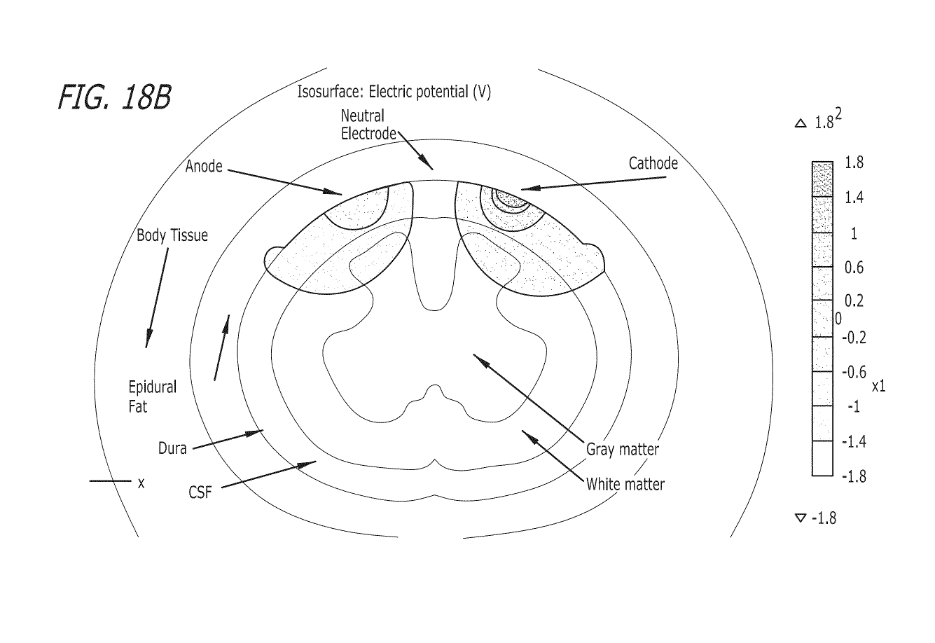

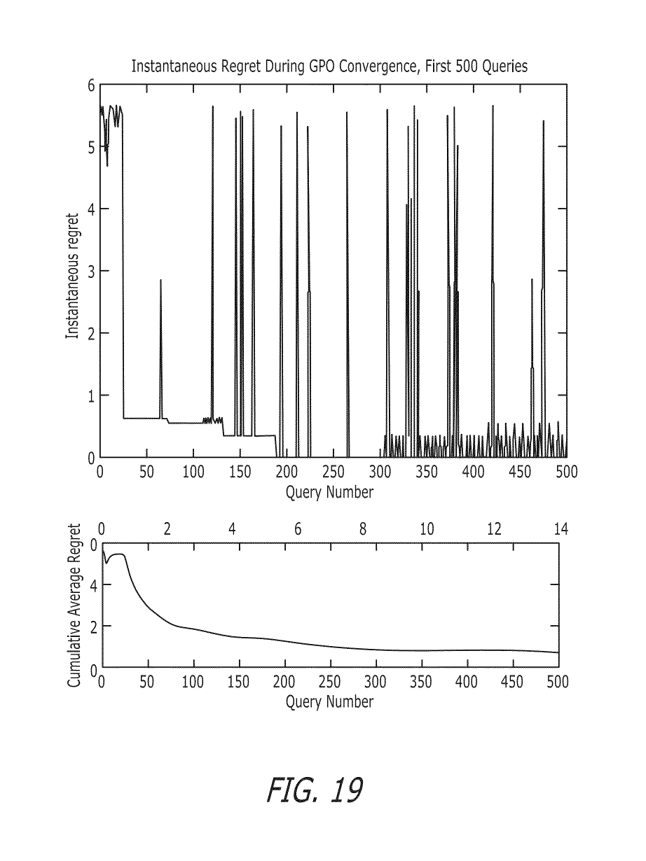

FIGS. 18A-18B depict a multi-compartment physical model of the electrical properties of mammalian spinal cord, along with a 27 electrode array placed in an epidural position. FIGS. 18A-18B also show the isopotential contours of the stimulating electric field for the 2-electrode stimulation example. FIG. 19 shows the instantaneous and average “regret” (a measure of the error in the learning algorithms search for optimal stimuli parameters) when the Gaussian Process Optimization algorithm summarized above is used to optimize the array stimulus pattern that excites neurons in the dorsal roots between segments L2 and S2 in the simulated spinal cord. The instantaneous regret performance shows that the learning algorithm rapidly finds better stimulating parameters, but also continually explores the stimulation space (the “bursts” in the graph of instantaneous regret correspond to excursions of the learning algorithm to regions of stimulus parameter space which were previously unknown, but which have been found to have poor performance).

### Use of Robotically Guided Training to Assist Recovery of Standing and Stepping

FIG. 2 shows that the use of physical training in combination with epidural stimulation and drug therapy produces better stepping behavior. Similarly, Example 1, herein, shows a similar effect of the combination of epidural stimulation and physical training/loading in a human subject.

While such physical manipulation can be facilitated by the use of trainers, e.g., as described above and in Example 1, in certain embodiments, the use of robotic devices and novel robotic control algorithms to guide and monitor the physical training process is contemplated. Robotic devices have been used successfully to train stepping and standing in complete spinal cord injured laboratory animals (Fong et al., J Neuroscience, 25(50): 11738-11747 (2005); de Leon et al., Brain Res Brain Res Rev., 40(1-3): 267-273 (2002); de Leon et al., J Neurophysiol., 182(1): 359-369 (1999)). However, recovery of effective patterns and levels of neuromuscular activity in humans with SCI (without epidural stimulation) as a result of training with a robotic device has not yet been as successful (Wernig, Arch Phys Med Rehabil., 86(12): 2385-2386 (2005); author reply 2386-2387).

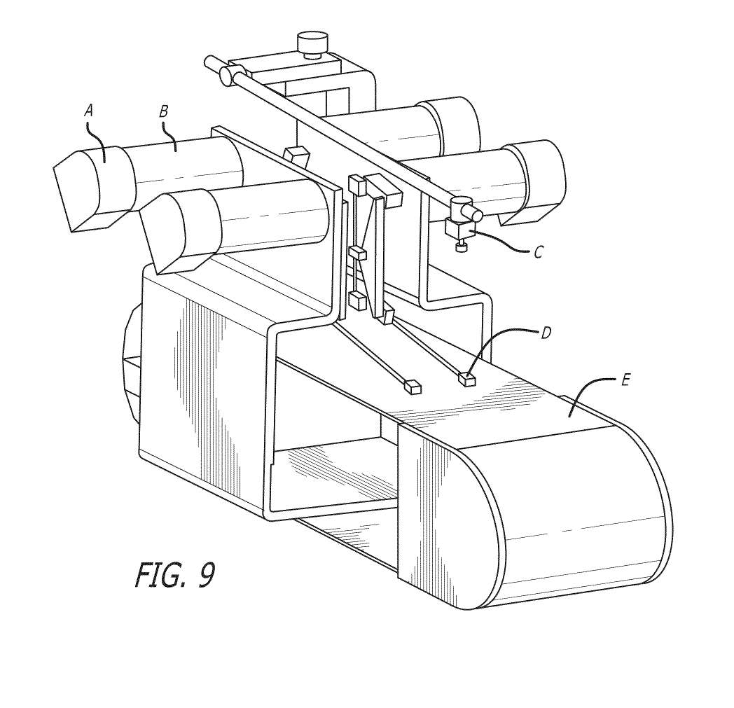

It is contemplated that “assist-as-needed” control algorithms that mimic the behavior of human therapists during weight supported treadmill step training of human SCI patients can be utilized. When the limb kinematics of the SCI patient are poor, the therapists provides a large amount of physical bias to force the limbs to follow a more normal stepping pattern, as well as cutaneous sensory input to trigger reflex responses. When the limbs are moving close to a normal stepping pattern, the therapist provides little physical bias or sensory input to the patient. We implemented these algorithms on the robot of FIG. 9, and found that even primitive assist-as-needed algorithms provide significant improvement in the rate and quality of step recovery. In this robotic device, lightweight low-friction robot arms guide the motions of the ankles of a weight-supported spinalized animal (mouse or rat) as it steps at various speeds on the moving treadmill. Because of the arms' low mass, they can also be used in a passive mode for testing locomotion ability—the movements of the animal's ankles are recorded by the robot as it attempts to walk on the treadmill (see, e.g., Cai, et al., Proc. Int. Conference Rehab. Robotics., 9: 575-579 (2005)).

### Pharmacological Facilitation of Stepping, Standing, Reaching and Grasping

In certain embodiments, the methods described herein are used in conjunction with various pharmacological agents. In particular, the use of various serotonergic, and/or dopaminergic, and/or noradrenergic and/or GABAergic, and/or glycinergic drugs, particularly drugs that have been demonstrated to be effective in facilitating stepping in animals is contemplated. These agents can be used in combination with epidural stimulation and physical therapy as described above. This combined approach can help to put the spinal cord (below the site of lesion) in an optimal physiological state for controlling a range of lower and upper limb movements.

In certain embodiments, the drugs are administered systemically, while in other embodiments, the drugs are administered locally, e.g., to particular regions of the spinal cord. Drugs that modulate the excitability of the spinal neuromotor networks are combinations of noradrenergic, serotonergic, GABAergic, and glycinergic receptor agonists and antagonists. Illustrative pharmacological agents include, but are not limited to agonists and antagonists to one or more combinations of serotonergic: 5-HT1A, 5-HT2A, 5-HT3, and 5HT7 receptors; to noradrenergic alpha1 and 2 receptors; and to dopaminergic D1 and D2 receptors (see, e.g., Table 1).

TABLE 1
Illustrative pharmacological agents.

| | | | | | |
|---|---|---|---|---|---|
| | | | | Optimal | Range of |
| | | | | Concen- | tested con- |
| | | | | tration | centrations |
| Name | Target | Action | Route | (mg/Kg) | (mg/Kg) |

Serotonergic receptor systems

| | | | | | |
|---|---|---|---|---|---|
| 8-OHDPAT | 5-HT1A7 | Agonist | S.C. | 0.05 | 0.045-0.3 |
| Way 100.635 | 5-HT1A | Antagonist | I.P. | 0.5 | 0.4-1.5 |
| Quipazine | 5-HT2A/C | Agonist | I.P. | 0.2 | 0.18-0.6 |
| Ketanserin | 5-HT2A/C | Antagonist | I.P. | 3 | 1.5-6.0 |
| SR 57227A | 5-HT3 | Agonist | I.P. | 1.5 | 1.3-1.7 |
| Ondanesetron | 5-HT3 | Antagonist | I.P. | 3 | 1.4-7.0 |
| SB 269970 | 5-HT7 | Antagonist | I.P. | 7 | 2.0-10.0 |

Noradrenergic receptor systems

| | | | | | |
|---|---|---|---|---|---|
| Methoxamine | Alpha1 | Agonist | I.P. | 2.5 | 1.5-4.5 |
| Prazosin | Alpha1 | Antagonist | I.P. | 3 | 1.8-3.0 |
| Clonidine | Alpha2 | Agonist | I.P. | 0.5 | 0.2-1.5 |
| Yohimbine | Alpha2 | Antagonist | I.P. | 0.4 | 0.3-0.6 |

Dopaminergic receptor systems

| | | | | | |
|---|---|---|---|---|---|
| SKF-81297 | D1-like | Agonist | I.P. | 0.2 | 0.15-0.6 |
| SCH-23390 | D1-like | Antagonist | I.P. | 0.15 | 0.1-0.75 |
| Quinpirole | D2-like | Agonist | I.P. | 0.3 | 0.15-0.3 |
| Eticlopride | D2-like | Antagonist | I.P. | 1.8 | 0.9-1.8 |

The foregoing embodiments are intended to be illustrative and not limiting. Using the teachings and examples provided herein, numerous variations on the methods and devices described herein will be available to one of ordinary skill in the art.

### EXAMPLES

The following examples are offered to illustrate, but not to limit the claimed invention.

### Example 1

### Epidural Stimulation of the Lumbosacral Spinal Cord Enables Independent Standing, Voluntary Movement, and Assisted Stepping in a Paraplegic Human

This example demonstrates that the human spinal cord circuitry has the ability to generate postural and locomotor patterns without supraspinal motor input. This capability and voluntary movement can be manifested when the excitability of these networks is modulated by epidural stimulation at a level that enables proprioceptive input to provide a source of neural control to elicit the motor pattern appropriate for the task.

### INTRODUCTION

The mammalian spinal cord can generate locomotor output in the absence of input from the brain. See Grillner S., Neurobiological bases of rhythmic motor acts in vertebrates, Science, 228:143-149 (1985); and Rossignol S, Barriere G, Frigon A, Barthelemy D, Bouyer L, Provencher J, et al., Plasticity of locomotor sensorimotor interactions after peripheral and/or spinal lesions, Brain Res Rev, 57(1):228-240 (January 2008). This capability has been attributed to the phenomenon of central pattern generation. See Grillner S, Wallen Peter, Central pattern generators for locomotion, with special reference to vertebrates, Ann Rev Neurosci, 8:233-261 (1985); and Grillner S, Zangger P., On the central generation of locomotion in the low spinal cat, Exp Brain Res, 34:241-261 (1979). Functional standing and stepping can be executed by cats with complete transection of the spinal cord when sensory input is provided to the lumbosacral locomotor pattern generator circuitry. See de Leon R D, Hodgson J A, Roy R R, Edgerton V R., Locomotor capacity attributable to step training versus spontaneous recovery after spinalization in adult cats, J Neurophysiol, 79:1329-1340 (1998); and Barbeau H, and Rossignol S., Recovery of locomotion after chronic spinalization in the adult cat, Brain Res, 412:84-95 (1987). Spinal cats can learn to stand, fully supporting their hindquarters, and to step over a range of speeds and load-bearing levels with task specific training. Adult spinally transected rats, unlike cats, can generate stepping only with additional combined interventions of locomotor training, pharmacological intervention, and/or epidural stimulation. See Courtine G, Gerasimenko Y, van den BR, Yew A, Musienko P, Zhong H, et al., Transformation of nonfunctional spinal circuits into functional states after the loss of brain input, Nat Neurosci, 12(10):1333-1342 (October 2009); and Ichiyama R M, Courtine G, Gerasimenko Y P, Yang G J, van den B R, Lavrov I A, et al., Step training reinforces specific spinal locomotor circuitry in adult spinal rats, J Neurosci, 16; 28(29):7370-7375 (July 2008). These observations demonstrate a level of automaticity sufficient to generate locomotion without any supraspinal influence. This evidence leads to the hypothesis that if similar spinal circuits exist in humans then electrically stimulating the lumbosacral spinal cord epidurally should be able to facilitate standing and stepping in an individual with a motor complete spinal cord injury.

Although, rhythmic motor patterns of the legs have been observed, 12-15 sustained independent, full weight-bearing standing and stepping has not been reported in humans after complete motor paralysis. See Calancie B., Spinal myoclonus after spinal cord injury, J Spinal Cord Med, 29:413-424 (2006); Dimitrijevic M R, Gerasimenko Y, Pinter M M, Evidence for a spinal central pattern generator in humans, Ann NY Acad Sci, 16; 860:360-376 (November 1998); Kuhn R A. Functional capacity of the isolated human spinal cord, Brain, 73(1):1-51 (1950); and Nadeau S, Jacquemin G, Fournier C, Lamarre Y, Rossignol S., Spontaneous motor rhythms of the back and legs in a patient with a complete spinal cord transection, Neurorehabil Neural Repair, 24(4):377-383 (May 2010). However, after a motor incomplete SCI functional improvements occur with intense locomotor training and with epidural stimulation. See Wemig A, and Muller S., Laufband locomotion with body weight support improved walking in persons with severe spinal cord injuries, Para; 30:229-238 (1992); Wemig A, Nanassy A, Muller S., Maintenance of locomotor abilities following Laufband (treadmill) therapy in para- and tetraplegic persons: follow-up studies, Spinal Cord, 36:744-749 (1998); and Herman R, He J, D'Luzansky S, Willis W, Dilli S., Spinal cord stimulation facilitates functional walking in a chronic, incomplete spinal cord injured. Spinal Cord, 40(2):65-68 (February 2002). Rhythmic efferent activity timed to the step cycle, however, can occur during manually facilitated stepping and bilateral tonic activity can occur during partial weight bearing standing in individuals with a clinically complete SCI after extensive task specific training. See Dietz V, Colombo G, Jensen L., Locomotor activity in spinal man, The Lancet, 344:1260-1263 (1994); Harkema S J, Hurley S L, Patel U K, Requejo P S, Dobkin B H, Edgerton V R, Human lumbosacral spinal cord interprets loading during stepping, J Neurophysiol, 77(2):797-811 (1997); and Harkema S J, Plasticity of interneuronal networks of the functionally isolated human spinal cord, Brain Res Rev, 57(1):255-264 (January 2008). Rhythmic and tonic motor patterns of the legs have been induced via epidural stimulation in humans after motor complete SCI while lying supine. See Dimitrijevic M R, Gerasimenko Y, Pinter M M, Evidence for a spinal central pattern generator in humans, Ann NY Acad Sci, 16; 860:360-376 (November 1998); Gerasimenko Y, Daniel O, Regnaux J, Combeaud M, Bussel B., Mechanisms of locomotor activity generation under epidural spinal cord stimulation, In: Dengler R, Kossev A, editors, Washington, D.C.: IOS Press, p. 164-171 (2001); and Minassian K, Jilge B, Rattay F, Pinter M M, Binder H, Gerstenbrand F, et al., Stepping-like movements in humans with complete spinal cord injury induced by epidural stimulation of the lumbar cord: electromyographic study of compound muscle action potentials, Spinal Cord, 42(7):401-416 (July 2004). This suggests that spinal circuitry for locomotion is present in the human but cannot functionally execute these tasks without some level of excitability from supraspinal centers that may be present after incomplete SCI.

We hypothesized that tonic epidural spinal cord stimulation can modulate the human spinal circuitry into a physiological state that enables sensory input derived from standing and stepping movements to serve as a source of neural control to perform these tasks. We observed that the spinal circuitry was able to generate independent standing in response to task specific sensory cues in the presence of epidural stimulation in a paraplegic subject with a motor complete spinal cord injury. Stepping-like patterns were also generated with epidural stimulation with the subject on a treadmill using body weight support and manual facilitation. The subject also regained some voluntary control of the legs seven months post implantation. We have used epidural stimulation to substitute for descending signals that normally come from the brain to modulate the physiological state of the spinal networks and the sensory information can be used as a source of neural control of the motor task. Unexpectedly, clinical assessments indicated improvements in other physiological functions including bladder, sexual function and temperature regulation.

### Methods

### Clinical Characteristics Prior to Implantation.

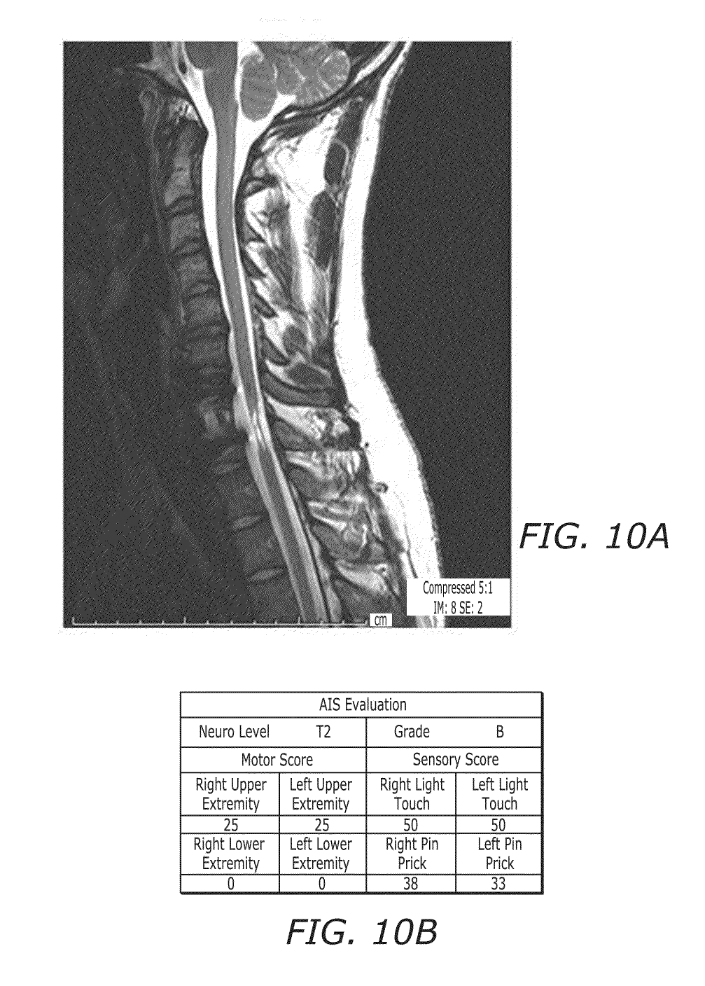

The subject is a 23 year old man who had been struck by a motor vehicle 3.4 years prior to implantation. He sustained a C7-T1 subluxation with injury to the lower cervical and upper thoracic spinal cord. Neurological examination revealed paraplegia. The triceps and intrinsic hand muscles exhibited voluntary contraction but were weak. He had no contraction of trunk or leg muscles. He was treated emergently with reduction of the subluxation by anterior interbody fusion and instrumentation. Magnetic resonance imaging of the injury site obtained prior to implantation revealed myelomalacia and atrophy of the cord segment adjacent to the T1 vertebral body (see FIG. 10A).

Prior to the lumbosacral epidural implantation his neurological deficit was classified using the American Spinal Injury Association (ASIA) impairment scale (AIS) as ASIA B (pinprick and light-touch present below the lesion). Marino R J, Barros T, Biering-Sorensen F, Burns S P, Donovan W H, Graves D E, et al., International standards for neurological classification of spinal cord injury, J Spinal Cord Med, 26 Suppl 1:S50-S56 (2003). He had no motor function of trunk or leg muscles, a flaccid anal sphincter, and no voluntary bladder contraction (see FIG. 10B). Sensation was abnormal below C7.

Somatosensory evoked potentials showed bilateral delay of cortical responses from posterior tibial nerve stimulation. Latencies of sensory evoked potentials recorded at Erb's point, cervical, and contralateral cortical sites in response to median nerve stimulation at the wrist were within normal ranges. Lower extremity nerve conduction studies were normal. No response was elicited from leg muscles by transcranial magnetic stimulation of the motor cortex using a butterfly coil centered over Cz. He was unable to stand or walk independently or voluntarily move his legs despite standard-of-care rehabilitation and additional intensive locomotor training.

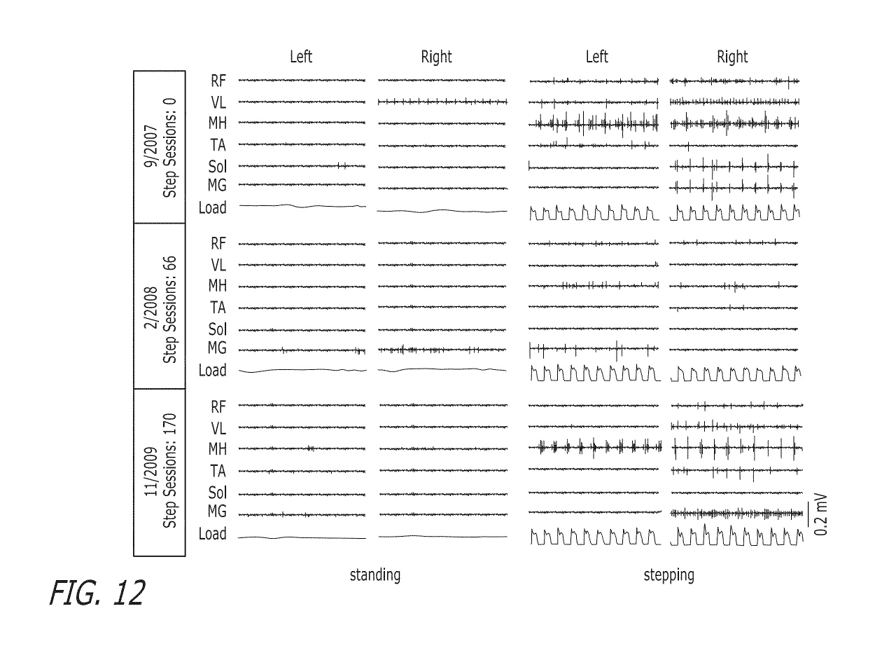

The research subject signed an informed consent for electrode implantation, stimulation, and physiological monitoring studies that was approved by the University of Louisville and the University of California, Los Angeles Institutional Review Boards. To be certain there was no remaining potential for standing and walking, prior to the electrode implantation, the participant received 170 locomotor training sessions over a period of 26 months using body weight support on a treadmill with manual facilitation resulting in 108 hours of step training and 54 hours of stand training with no detectable change in EMG activity (see FIG. 12). During standing, throughout training no observable EMG was evident. During assisted stepping, sporadic EMG activity was observed in the lower leg muscles, most often in the medial hamstrings, however, was never observed EMG activity in all muscles bilaterally. No detectable improvement in EMG was noted over the course of the training.

### Surgical Implantation of Electrode Array and Stimulator

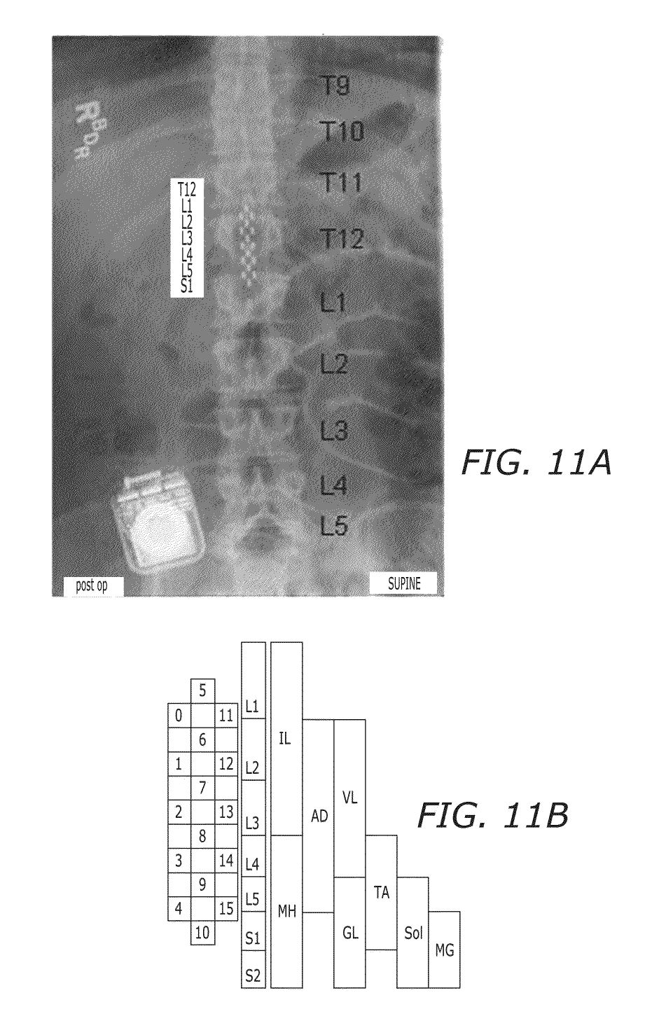

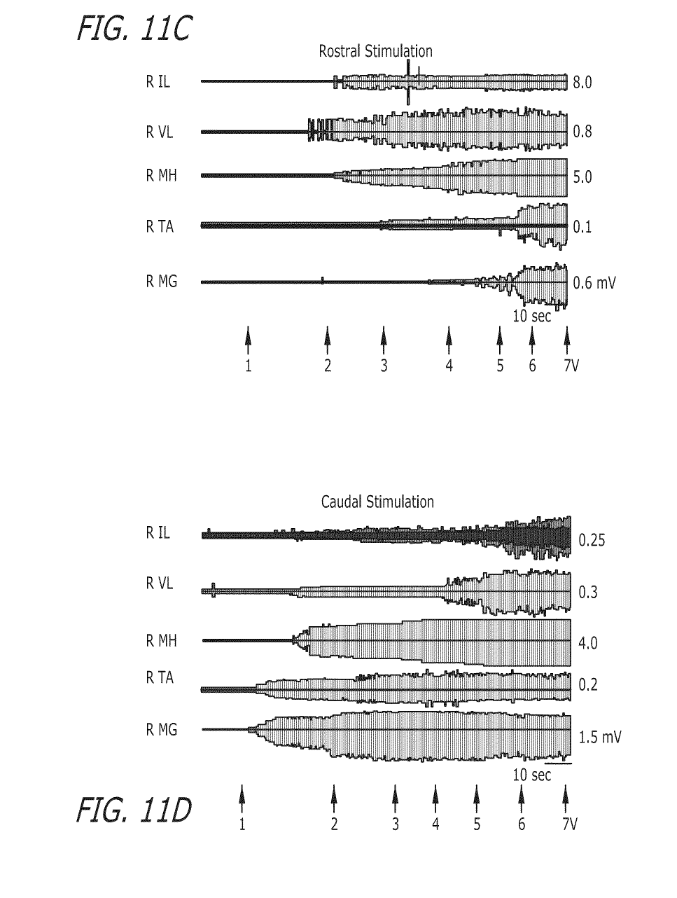

An epidural spinal cord stimulation unit (Medtronics, Restore Advanced) was used to electrically stimulate the lumbar-sacral enlargement. A 16-electrode array was implanted epidurally under fluoroscopic control at T11-L1 over lumbosacral spinal cord segments L1-S1 (see FIG. 11A). The location of the electrode array was evaluated and adjusted during surgery with fluoroscopy and electrophysiologically with EMG recorded from leg muscles. See Murg M, Binder H, Dimitrijevic M R, Epidural electric stimulation of posterior structures of the human lumbar spinal cord: 1. muscle twitches—a functional method to define the site of stimulation, Spinal Cord, 38:394-402 (2000). EMG responses were elicited by epidural stimulation at 2 Hz during a sequence of increasing voltages and specific electrode configurations to determine threshold of muscle activation and amplitude of the response. A midline stimulation configuration was followed using one cathode and one anode electrode, with each electrode pair being 6 mm apart. Multiple stimulation combinations were performed ranging from most rostral to most caudal positions. Symmetry was also tested by using left and right side electrodes within the array. The electrode lead was tunneled to a subcutaneous abdominal pouch where the pulse generator was implanted. Two weeks after implantation the position of the array was reconfirmed with the subject lying supine using the same stimulation protocols (see FIGS. 11C-11D).

### Experimental Design

Stimulation parameters were systematically evaluated to identify the optimal stimulation parameters for generating efferent patterns for standing and stepping. Stimulation of the spinal cord was carried out during sessions lasting up to 250 minutes in which physiological parameters were measured. The total duration of stimulation during each experimental session ranged from 40 minutes to 120 minutes. Stimulation amplitudes ranged from 0.5 V to 10.0 V and stimulation frequencies from 5 to 40 Hz using either a 210 or 450 μs pulse width. The optimal configurations for standing were those with which sustainable tonic co-activation were evoked; for stepping optimal configurations were those in which rhythmic activity was present with alternation of right and left leg and intralimb flexors and extensors. EMG activity of 14 lower extremity muscles and hip, knee, and ankle joint angles were measured.

During experimental sessions on the treadmill, level of body weight support (Innoventor, St. Louis, Mo.) and amount of body weight load were also measured. Trainers provided manual facilitation, when needed, distal to the patella during the stance phase, and at the popliteal fossa and anterior distal tibia for foot clearance during the swing phase and at the pelvis for stabilization and weight shifting during stepping. Stand training was performed using a custom-made standing device designed to provide full weight-bearing and pelvis support. The device included vertical and horizontal bars positioned about (or surrounding) the subject to allow him to assist balance. Bungees were attached to the device to provide support only if the knees or hips flexed beyond the normal standing posture. The total duration of stimulation during each session averaged 44 minutes (sessions 1-34) and 60 minutes (sessions 35-80). Epidural stimulation was not provided outside laboratory sessions. The subject attempted to stand for 60 minutes during each training session. To optimize independent standing stimulation parameters (electrode configuration, voltage and frequency) were modified approximately once per week.

During sitting, stimulation voltage was increased to a desired level. This voltage was kept constant as the subject went from sitting to standing and throughout the standing bout. The subject initiated the sit to stand transition by positioning his feet shoulder width apart and shifting his weight forward to begin loading the legs. The subject used the bars of the standing device during the transition phase to balance and to partially pull himself into a standing position. Trainers positioned at the pelvis and knees assisted as needed during the sit to stand transition. Elastic bungees posterior to the pelvis were set by one of the trainers after the subject achieved full-weight bearing standing. These bungees helped the subject sustain appropriate pelvic tilt and position and allowed him to safely stand with minimal assistance.

During the standing bout, one trainer assisted the subject by applying posteriorly directed gentle pressure at the patellar tendon as necessary to maintain knee extension. The subject was encouraged to stand for as long as possible throughout the session.

Seated resting periods occurred when requested by the subject and reduced in frequency and duration as the training progressed. No stimulation was provided during the rest periods.

During the first stand session, the subject required 7 breaks (stand time: 60 min; rest time 67 minutes). By session 35, the subject was able to stand for 1 bout lasting a full 60 minutes. The total duration of stimulation averaged across all sessions was 54±13 minutes per session.

### Data Acquisition

EMG, joint angles, footswitch, ground reaction forces and BWS data were collected at 2,000 Hz using a 32-channel hard-wired AD board and custom-written acquisition software (National Instruments, Austin, Tex.). Bilateral EMG (Motion Lab Systems, Baton Rouge, La.) from the soleus, medial gastrocnemius, tibialis anterior, medial hamstrings, quadriceps, and gluteus maximus muscles was recorded using bipolar surface electrodes with fixed inter-electrode distance. Harkema S J, Hurley S L, Patel U K, Requejo P S, Dobkin B H, Edgerton V R, Human lumbosacral spinal cord interprets loading during stepping, J Neurophysiol, 77(2):797-811 (1997); and Beres-Jones J A, Johnson T D, Harkema S J, Clonus after human spinal cord injury cannot be attributed solely to recurrent muscle-tendon stretch, Exp Brain Res, 149(2):222-236 (March 2003). Bilateral EMG from the iliopsoas was recorded with fine-wire electrodes. Two surface electrodes placed symmetrically lateral to the electrode array incision site over the paraspinal muscles were used to record the stimulation artifact. Hip, knee, and ankle joint angles were acquired using a high speed passive marker motion capture system (Motion Analysis, Santa Rosa, Calif.). Ground reaction forces were collected using shoe-insole pressure sensors FSCAN or HRMAT (TEKSCAN, Boston, Mass.).

### Results

The patient was always aware when the stimulation was on, with the most common sensation being a tingling feeling localized to the thoraco-lumbar electrode implantation site. There was a similar sensation in those muscles that were targeted for activation. Parasthesias were also routinely perceived in the trunk, hips, and legs and varied according to the intensity of stimulation, however were never at a level that produced discomfort or pain and never precluded the use of epidural stimulation.

### EMG Activity with Epidural Stimulation for Standing

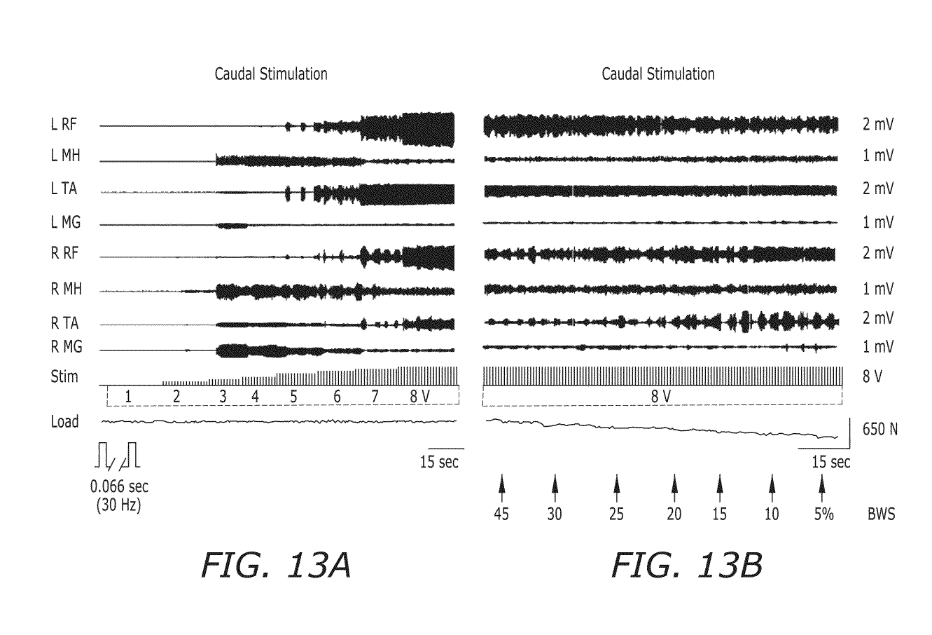

Epidural stimulation at 15 Hz and 8 V of the caudal segments (L5-S1) of the spinal cord combined with sensory information related to bilateral extension and loading was sufficient to generate standing on day five of stimulation (see FIG. 13). Standing without manual facilitation at the legs was achieved using stimulation (15 Hz, 8 V) with 65% body weight support (see FIG. 13, panel A). The subject was able to sustain standing without any manual facilitation while the level of body weight support was progressively reduced to full weight-bearing (see FIG. 13, panel B).

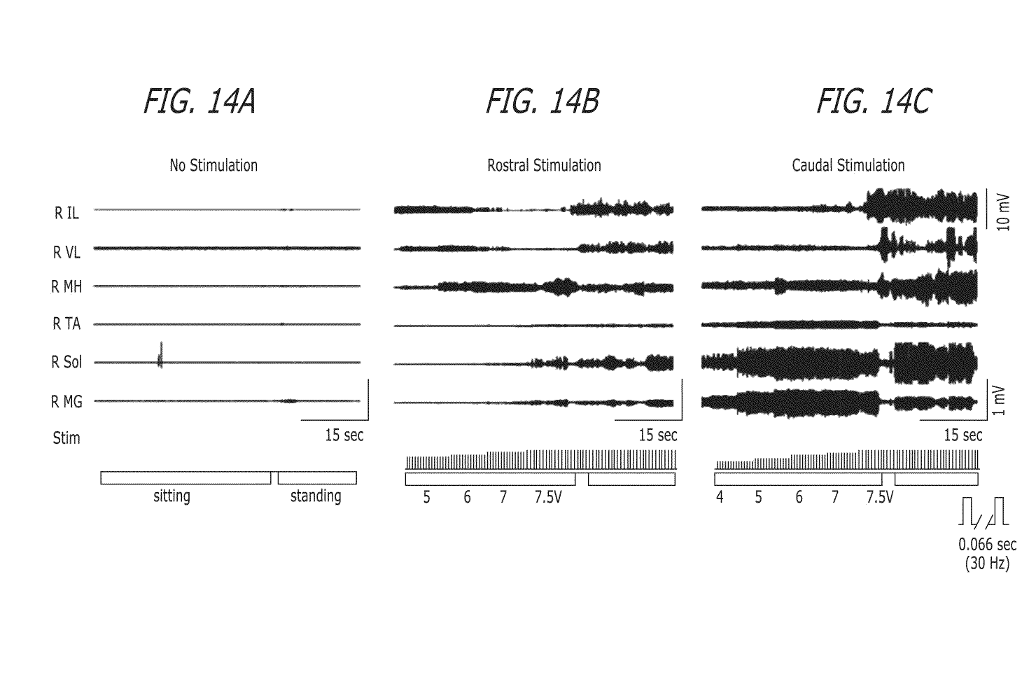

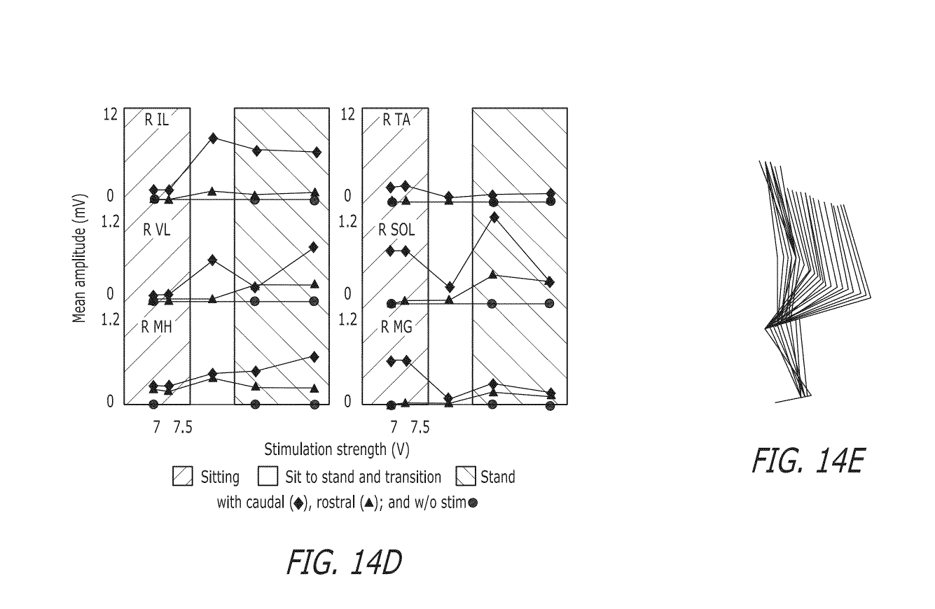

Transitioning from sitting to standing without body weight support altered the EMG activity during rostral or caudal epidural stimulation even though the parameters remained constant (see FIG. 14). When loading of the legs was initiated, the EMG activity increased dramatically and was sufficient to support the subject's body weight with minimal assistance required by the trainers. During this transition, the stimulation remained constant using the same location, frequency, and intensity parameters (FIG. 14, panels B-E). The EMG activity was also modulated by the site and intensity of stimulation. The EMG activity was dependent on the site and intensity of stimulation with the caudal (L5-S1) stimulation at higher intensities resulting in the most optimal motor pattern for standing (see FIG. 14, panels A-C). During caudal stimulation, there was a more dramatic increase in the EMG amplitude bilaterally in the more proximal muscles while EMG of the more distal muscles was initially markedly reduced (see FIG. 14, panels C and E). Once standing was achieved, there was more co-contraction of both flexors and extensors and proximal and distal muscles with stimulation.

Postural Responses and Independent Standing with Epidural Stimulation

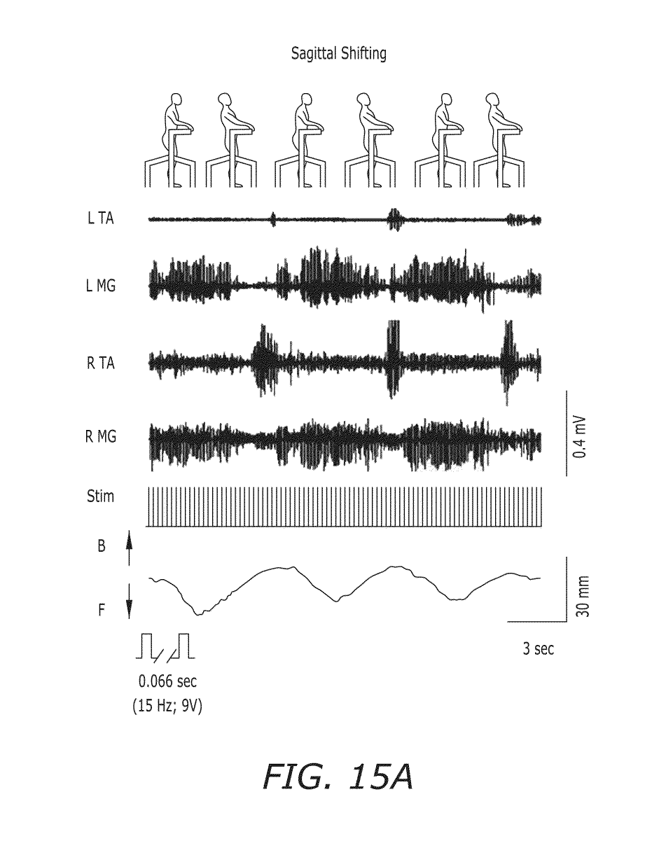

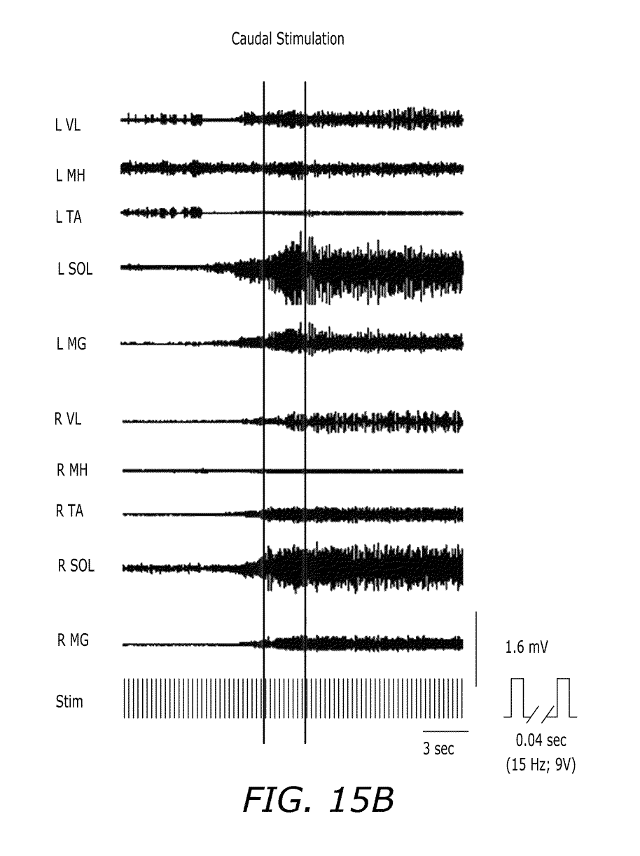

Postural responses were observed in the leg EMG activity when the subject voluntarily shifted his center of gravity sagittally while standing with epidural stimulation and intermittent manual assistance (see FIG. 15, panel A). The EMG burst of the medial gastrocnemius increased with forward deviation, whereas backward deviation induced EMG bursts in the tibialis anterior. Independent standing bouts with tonic bilateral EMG activity routinely occurred for several continuous minutes and increased in frequency and duration as stand training progressed (see FIG. 15, panel B). After 80 sessions, the subject could initiate and maintain continuous independent standing (maximum 4.25 min) with bilateral tonic EMG activity (see FIG. 15, panel B). Oscillatory patterns, often clonic-like, emerged during the latter part of the periods of independent standing and then were followed by little or no EMG activity that corresponded with the loss of independence (requiring a return to manually facilitated standing). These periods of independent standing were repeated during the 60-minute standing sessions.

Thus, independent standing occurred when using stimulation having parameters selected (e.g., optimized) to facilitate standing while providing bilateral load-bearing proprioceptive input.

Locomotor Patterns with Epidural Stimulation

For stepping, epidural stimulation at 30-40 Hz and task-specific sensory cues were used to generate locomotor-like patterns. Sensory cues from manually facilitated stepping included load alternation and leg positioning with appropriate kinematics of the hips, knees, and ankles timed to the step cycle. Stepping with BWST without epidural stimulation produced little or no EMG activity (see FIG. 16, panel A). Stepping with BWST and manual facilitation in conjunction with caudal epidural stimulation resulted in an oscillatory EMG pattern in flexors and extensors (see FIG. 16, panel B). The afferent feedback determined the motor efferent pattern (see FIG. 16, panels C and D). The EMG activity in the legs was dramatically different depending on the loading and kinematic patterns when using the identical stimulation parameters. Oscillatory EMG patterns were evident only when alternating loading and flexion and extension of the lower limbs occurred (see FIG. 16, panels C and D).

### Voluntary Control of Leg Movement

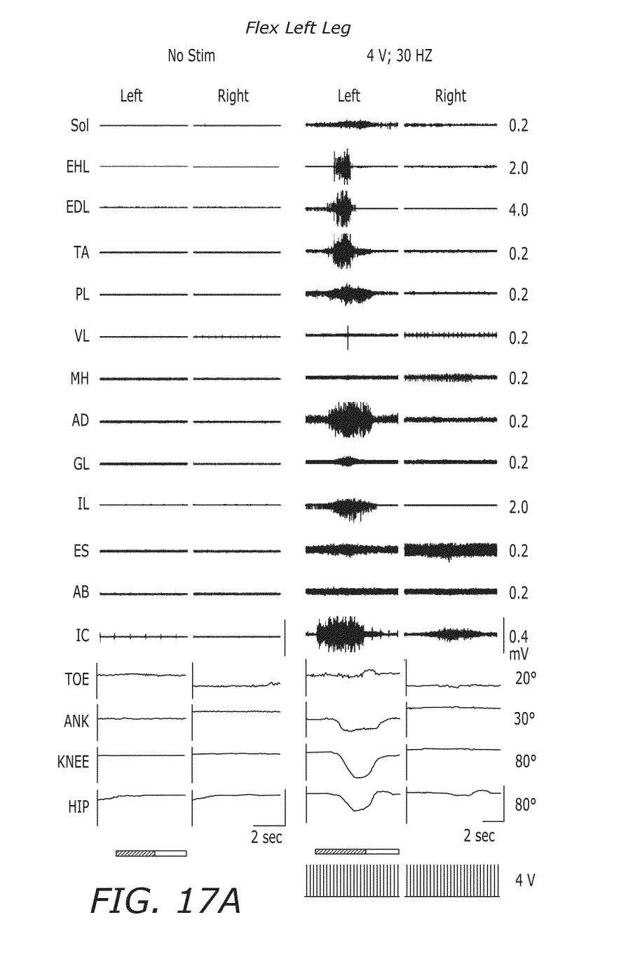

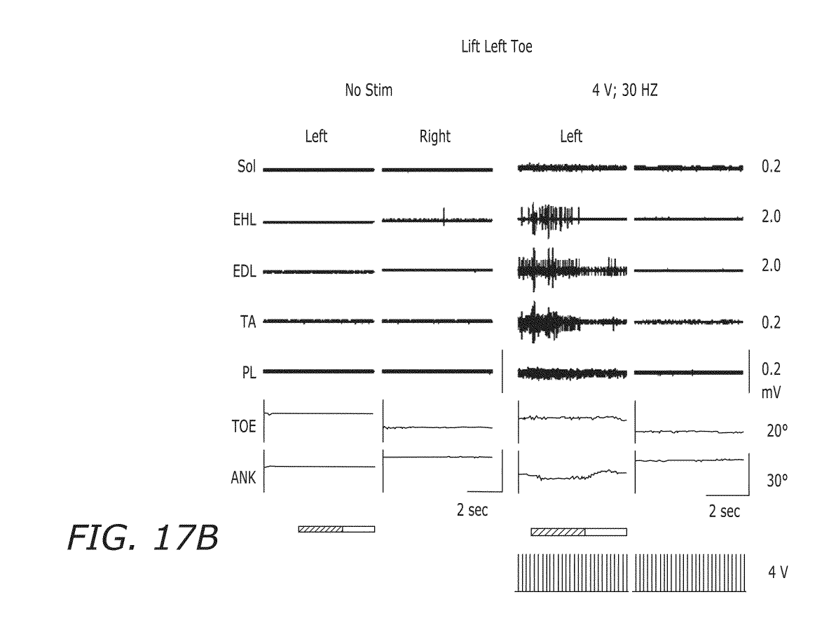

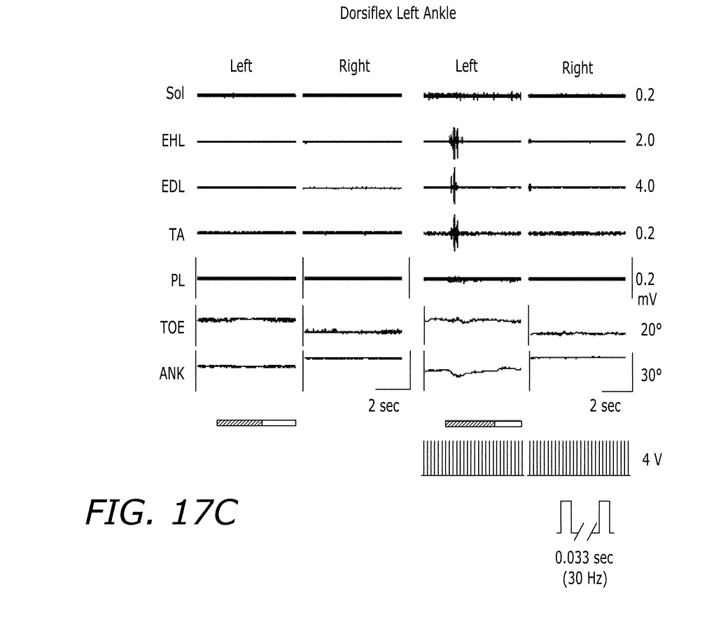

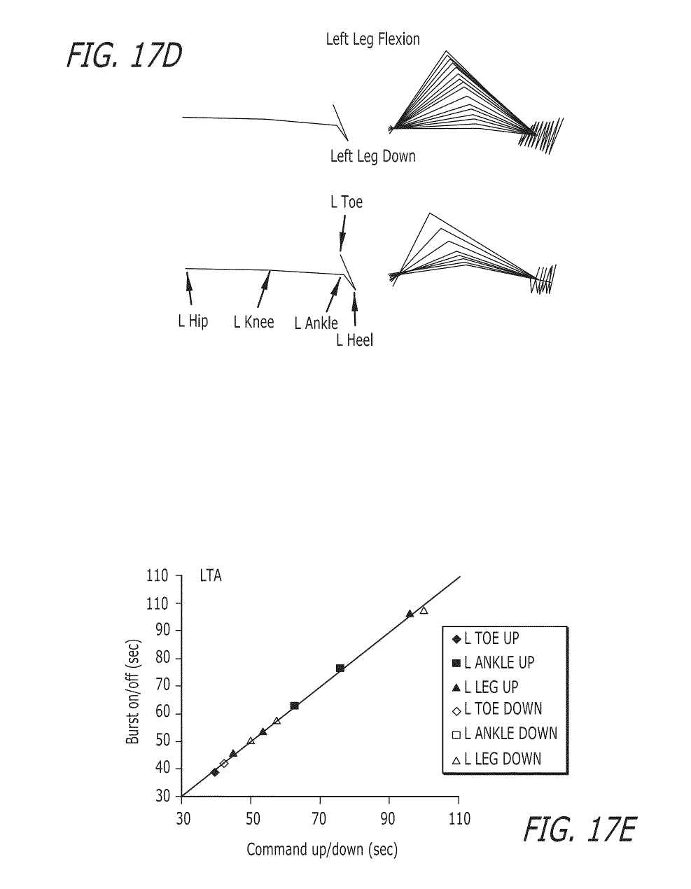

Voluntary (or supraspinal) control of the toe extension, ankle flexation, and leg flexion emerged only in the presence of epidural stimulation (see FIG. 17) seven months after the epidural implant that included 80 stand training sessions with epidural stimulation. Voluntary movement was observed in both limbs. However, the epidural stimulation parameters were different for each leg and technical limitations of the stimulator prevented simultaneous movements of the legs bilaterally. When the subject was instructed to flex (draw the leg upward) the toe extended, the ankle dorsi-flexed and the hip and knee flexed with the appropriate muscle activation. When instructed to dorsi-flex the ankle, the foot moved upward with tibialis anterior activation. When instructed to extend the great toe, the toe moved upward with activation of the extensor hallicus longus. For each task, the muscle activation was specific for the movement and the timing of activation was closely linked to the verbal commands (see FIGS. 17C-17E). The subject could consciously activate the appropriate muscles for the intended movement, and the timing of activation was closely linked to the verbal commands (see FIG. 17E). The ability to selectively activate different motor pools demonstrates an important feature of voluntary motor control.

Thus, locomotor-like patterns were observed when stimulation parameters were selected (e.g., optimized) to facilitate stepping. Further, seven months after implantation, the subject recovered supraspinal control of certain leg movements, but only during epidural stimulation.

### Subject's Perspective

Given the uniqueness of the epidural stimulation procedures and the unusual level of commitment of the subject to the objectives of the study, the research team asked the subject his perspective on a range of highly personal topics related to changes in his health and daily living after compared to before the implant.

Interpretation of these responses should take into account that the subject received extensive rehabilitation for 170 sessions immediately before the implant. Specifically, the subject provided the following responses as to how (other than demanding so much of his time) the experience affected the specified aspect of his life:

1. sleep patterns: I am sleeping more soundly, and am able to reach a deeper level of sleep (the dream phase) almost every night. I have also noticed that I need more sleep, at least 10 hours a night and sometimes more after a hard or draining workout.

2. daily activity patterns: Besides the issue of being tired from the workouts, I have had more over all energy. I have been more active during the days than before the implant. This has improved since the first few workouts after the surgery, since at first I could not do anything and even had trouble transferring after workouts, but this has continuously gotten better every day.

3. bladder or bowel function: In terms of my bladder, I've been able to empty more often on my own, on command, without a catheter. So far I've had no infections as well. In terms of my bowel function, I'm more regular.

4. sensory function: I've been able to feel more sharp and dull sensations in places where I wasn't able to before the surgery, such as through my stomach and legs. Also I'm having better sensation with light touch throughout my midsection and legs. Refer to most recent ASIA exam where I had mostly zeros before surgery and now have mostly ones.

5. severity and frequency and timing of spasticity: My spasticity has increased only when lying down.

6. frequency and kind of medical care needed: Other than when my stitches opened shortly after surgery no medical care has been needed since surgery.

7. sexual function: Erections have been stronger and more frequent and I am able to reach full orgasm occasionally. I had never before been able to do this before the surgery.

8. diet, appetite: I feel like I get hungrier after working out, but other than that no change.

9. body weight: I've gained about 9 kilograms since surgery.

10. observable changes in muscle: My leg muscles have increased by a few inches and I am able to see definition in my quads and calfs. My upper body (biceps, triceps, shoulders etc.) have also gained inches of muscle and I have not lifted a weight since surgery. My overall core has gotten stronger and more stable.

11. posture and stability when sitting: My posture has improved. I'm more stable and have less need to hold onto things to support myself.

12. skin lesions or sensitivity to infections: I have had no infections or skin lesions.

13. other functions: I feel healthier, I have better self-esteem and confidence. My legs are heavier and more dense.

### Clinical Impressions

With training and epidural stimulation, the subject had functional gains in bladder and sexual function, and temperature regulation. The subject has been able to voluntarily void with minimal residual volume, and reports normal sexual response and performance. The subject regained diaphoretic capability and ability to tolerate temperature extremes. In addition, a sense of well-being and increased self-esteem enabled more frequent social interactions. An eighteen percent gain in weight was associated with increased appetite and relative increase in lean body mass and decrease in total body fat as measured using aq DEXA scan.

### Discussion

We have used an epidurally implanted electrode array to modulate the physiological state of the spinal circuitry to enable independent standing in a human with a chronic motor complete spinal cord injury. The epidural stimulation did not induce standing by directly activating motor pools, but enabled motor function by engaging populations of interneurons that integrated load-bearing related proprioceptive input to coordinate motor pool activity. This phenomenon was observed within the first week of stimulation. Although motor pool activity occurred in the presence of epidural stimulation during sitting, the functional activity needed for standing required the proprioceptive information associated with load bearing positional changes. Dynamic changes in position during standing were accompanied by motor patterns needed to maintain upright posture without changes in the epidural stimulation parameters. Intensive task specific training combined with epidural stimulation extended the duration of periods of independent standing that could be initiated by the subject.

Robust, consistent rhythmic stepping-like activity emerged during stepping only when tonic epidural stimulation and weight-bearing associated proprioception was present. When standing, the same epidural stimulation parameters elicited primarily tonic bilateral activity; however when stepping it resulted in rhythmic alternating activity. Without being limited by theory, it is believed the epidural stimulation may activate dorsal root afferent fibers and, more likely at higher intensities, dorsal columns and additional spinal structures. The continuous stimulation enabled the spinal cord to process the sensory information that is closely linked to the desired functional task by modulating the physiological state of the spinal cord. This is of great clinical importance and it allows the intervention to become feasible since the task needed can be driven and controlled by intrinsic properties of the nervous system rather than an external control system.

Our study demonstrates that the sensory input can serve as the controller of the spinal circuitry during independent standing and assisted stepping when enabled by epidural stimulation in the absence of supraspinal input in humans.

The present results show that movements of several lower limb joints can be controlled voluntarily. In subjects with a motor incomplete spinal injury, a common phenomenon is the general loss of specificity of control of selected muscles, however, the voluntary nature of these reported movements are selective. See Maegele M, Muller S, Wernig A, Edgerton V R, Harkema S J, Recruitment of spinal motor pools during voluntary movements versus stepping after human spinal cord injury, J Neurotrauma, 19(10):1217-1229 (October 2002). In Example 1, the activated motor pools were appropriate for the intended movement. Two possible mechanisms that might explain this result include: 1) that the epidural stimulation provided excitation of lumbosacral interneurons and motoneurons (Jankowska E., Spinal interneuronal systems: identification, multifunctional character and reconfigurations in mammals, J Physiol, 533 (Pt 1):31-40 (May 15 2001)) which, combined with the weak excitatory activity of residual motor axons descending through the cervicothoracic injury, achieved a level of excitation that was sufficient to fire the motoneurons and/or 2) axonal regeneration or sprouting may have been induced via activity-dependent mechanisms occurring over a period of months. It is highly significant from a neurobiological as well as a clinical perspective that this voluntary control was manifested only in the presence of continuous tonic epidural stimulation. This demonstrates that by elevating the level of spinal interneuronal excitability to some critical, but sub-threshold level, voluntary movements can be regained. Dimitrijevic M R, Gerasimenko Y, Pinter M M, Evidence for a spinal central pattern generator in humans, Ann NY Acad Sci, 16; 860:360-376 (November 1998). These same mechanisms may also explain the improved autonomic function in bladder, sexual, vasomotor, and thermoregulatory activity that has been of benefit to the subject. The areas of lumbosacral spinal cord stimulated included at least parts of the neural circuits that regulate these autonomic functions and may have also resulted in activity-dependent changes. In other words, given that the broad areas of the lumbosacral spinal cord stimulated include at least parts of the neural circuits that regulate these autonomic functions, these changes might have been expected if the neural networks controlling these autonomic functions are activity-dependent.

These data demonstrate that humans have conserved spinal locomotor circuitry as found in other mammals that include: 1) transition from a low level activity state to one that can generate active standing in the presence of tonic epidural stimulation; 2) gate tonic electrically evoked responses according to the task specific sensory input, resulting in specific patterns of coordination within and between the motor pools; 3) use appropriate task specific sensory input to control the level and timing of neural excitation sufficient to generate independent standing and facilitate stepping; and 4) to mediate voluntarily initiated movement of the lower limbs in the presence of epidural stimulation. A higher level of improvement in motor function may be achieved with the addition of pharmacological agents not only in spinal cord injury but also with other neuromotor disorders. See Fuentes R, Petersson P, Siesser W B, Caron M G, Nicolelis M A, Spinal cord stimulation restores locomotion in animal models of Parkinson's disease, Science, 323(5921):1578-1582 (Mar. 20, 2009).

In Example 1, epidural stimulation of the human spinal cord circuitry combined with task specific proprioceptive input resulted in novel postural and locomotor patterns. After seven months of stimulation and stand training, supraspinally mediated movements of the legs were manifested only in the presence of epidural stimulation. Task specific training with epidural stimulation may have reactivated previously silent spared neural circuits or promoted plasticity. Thus, such interventions may provide a viable clinical approach for functional recovery after severe paralysis.

The above example supports the following. First, it is possible to stimulate the lumbosacral spinal cord with a modest, but sufficient level of intensity to enable the sensory input from the lower limbs to serve as a source of control of standing and to some degree of stepping. Second, the ability to stand for greater durations increases with daily stand training. Third, after months of stand training in the presence of epidural stimulation, there was sufficient supraspinal and spinal reorganization to enable conscious control of movements of the lower limbs. Fourth, extensive reorganization of supraspinal and spinal motor systems can occur in response to activity-dependent interventions in an individual with complete paralysis for more than 3 years after a lower cervical-upper thoracic spinal cord injury. None of these observations in a human subject with this severity of injury have been made previously.

### Example 2

### Sub-Threshold Spinal Cord Stimulation Facilitates Spontaneous Motor Activity

Electrical enabling motor control (eEmc) combined with spontaneous cage activity may increase the frequency and level of activation of the locomotor circuits in paralyzed rats. Spontaneous cage activity was recorded using a specially designed swivel connector to record EMG signals and an IR based camcorder to record video.

The spinal rats initially were very lethargic in their cages showing little movement. Without eEmc, the rats remained rather inactive with the torso rarely being elevated from the cage floor. When the rats used their forelimbs to move, the hindlimbs were extended and dragged behind with little or no flexion. In contrast, with eEmc the rats were highly active and the hindlimbs showed robust alternating flexion and extension resulting in step-like movements during forelimb-facilitated locomotion and often would stand using the sides of the cages as support. The mean and summed integrated EMG levels in both a hindlimb flexor and extensor muscle were higher with than without eEmc. eEmc, in combination with associated proprioceptive input, can modulate the spinal networks to significantly amplify the amount and robustness of spontaneous motor activity in paralyzed rats.

Here, modulation of the excitability of the lumbosacral region of the spinal cord via eEmc, combined with weak excitatory activity of descending axons that are not otherwise detectable, may volitionally achieve a level of excitation that is sufficient to activate the spinal motor circuits above the motor thresholds of a significant number of motoneurons among synergistic motor pools. In some embodiments, patients clinically diagnosed as having complete paralysis can use proprioceptive information combined with some input from descending motor signals (perhaps residual but functionally silent without eEmc) to activate spinal motor circuits, thus generating and controlling a range of motor functions via eEmc.

There is some spontaneous activity in the paralyzed muscles after a complete mid-thoracic spinal cord transection. The present experiment determind the feasibility of enhancing the amount of spontaneous cage activity of paralyzed muscles using sub-threshold intensities of stimulation via chronically implanted epidural electrodes placed over the lumbosacral spinal cord in adult spinal rats. We chose rats that had experienced a rehabilitation process to step on a treadmill for 6 weeks under the influence of eEmc because chronic step training engages and reinforces the locomotor networks that would potentially be activated during spontaneous cage activity. We determined the activity levels and movement patterns of the hindlimbs of rats having a complete spinal cord transection at a low thoracic level while in their home cages during 6-hr periods with and without continuous eEmc (40 Hz).

Delivered eEmc can modulate the spinal locomotor circuits such that the hindlimbs would be more active during periods with than without eEmc. This can have the effect of more frequently engaging those neural networks that control the routine, spontaneous postural and locomotor functions that are critical in defining the level of functionality after severe paralysis.

### Methods

### General Animal Procedures

Data were obtained from 4 adult female Sprague Dawley rats (270-300 g body weight). The rats were housed individually with food and water provided ad libitum. All survival surgical procedures were conducted under aseptic conditions and with the rats deeply anesthetized with isoflurane gas administered via facemask as needed. All procedures described below are in accordance with the National Institute of Health Guide for the Care and Use of Laboratory Animals and were approved by the Animal Research Committee at UCLA.

### Head Connector and Intramuscular EMG Electrode Implantation

A small incision was made at the midline of the skull. The muscles and fascia were retracted laterally, small grooves were made in the skull with a scalpel, and the skull was dried thoroughly. Two amphenol head connectors with Teflon-coated stainless steel wires (AS632, Cooner Wre, Chatsworth Calif.) were securely attached to the skull with screws and dental cement. Selected hindlimb muscles, i.e., the tibialis anterior (TA) and soleus (Sol), were implanted bilaterally with EMG recording electrodes as described in Roy R R, Hutchison D L, Pierotti D J, Hodgson J A, Edgerton V R: EMG patterns of rat ankle extensors and flexors during treadmill locomotion and swimming. J Appl Physiol 1991, 70:2522-2529. Skin and fascial incisions were made to expose the belly of each muscle. Two wires extending from the skull-mounted connector were routed subcutaneously to each muscle. The wires were inserted into the muscle belly using a 23-gauge needle and a small notch (˜0.5-1.0 mm) was removed from the insulation of each wire to expose the conductor and form the electrodes. The wires were secured in the belly of the muscle via a suture on the wire at its entrance into and exit from the muscle belly. The proper placement of the electrodes was verified during the surgery by stimulating through the head connector and post-mortem via dissection.

### Spinal Cord Transection and eEmc Electrode Implantation Procedures and Post-Surgical Animal Care

A partial laminectomy was performed at the T8-T9 vertebral level. A complete spinal cord transection to include the dura was performed at approximately the T8 spinal level using microscissors. Two surgeons verified the completeness of the transection by lifting the cut ends of the spinal cord and passing a glass probe through the lesion site. Gel foam was inserted into the gap created by the transection as a coagulant and to separate the cut ends of the spinal cord.

For eEmc electrode implantation, partial laminectomies were performed to expose the spinal cord at spinal levels L2 and S1. Two Teflon-coated stainless steel wires from the head connector were passed under the spinous processes and above the dura mater of the remaining vertebrae between the partial laminectomy sites. After removing a small portion (˜1 mm notch) of the Teflon coating and exposing the conductor on the surface facing the spinal cord, the electrodes were sutured to the dura mater at the midline of the spinal cord above and below the electrode sites using 8.0 Ethilon suture (Ethicon, New Brunswick, N.J.). Two common ground (indifferent EMG and stimulation grounds) wires (˜1 cm of the Teflon removed distally) were inserted subcutaneously in the mid-back region. All wires (for both EMG and eEmc) were coiled in the back region to provide stress relief.

All incision areas were irrigated liberally with warm, sterile saline. All surgical sites were closed in layers using 5.0 Vicryl (Ethicon, New Brunswick, N.J.) for all muscle and connective tissue layers and for the skin incisions in the hindlimbs and 4.0 Ethilon for the back skin incision. All closed incision sites were cleansed thoroughly with saline solution. Analgesia was provided by buprenex (0.5-1.0 mg/kg, s.c. 3 times/day). The analgesics were initiated before completion of the surgery and continued for a minimum of 3 days. The rats were allowed to fully recover from anesthesia in an incubator. The rats were housed individually in cages that had ample CareFresh bedding, and the bladders of the spinal rats were expressed manually 3 times daily for the first 2 weeks after surgery and 2 times daily thereafter. The hindlimbs of the spinal rats were moved passively through a full range of motion once per day to maintain joint mobility.

### Stimulation and Testing Procedures

The rats went through a bipedal step training rehabilitation process (20 min a day, 5 days a week) for 6 weeks under the influence of eEmc at 40 Hz between L2 and S1 at an intensity just above threshold using a body weight support system. Chronic step training was used because it engages and reinforces the locomotor networks that would potentially be activated during spontaneous cage activity.

The rats were tested under two conditions with and without eEmc at 40 Hz between L2 and S1 at 6 weeks post-injury: 1) during bipedal stepping on a specially designed motor-driven rodent treadmill using a body weight support system and 2) during spontaneous cage activity. The eEmc during treadmill locomotion was set just above threshold. The threshold for eliciting a muscle twitch and corresponding time linked EMG response (soleus was used as the reference muscle) was between 1.8 to 2 V for all rats. The subthreshold level then was set at 20% below the motor threshold, i.e., between 1.4 and 1.6 V, during the recording of spontaneous cage activity.

The spontaneous activity levels of the spinal rats were determined in their home cage. The head connector was connected via cables to a set of amplifiers and a stimulator. A swivel arrangement was attached to the cables near the head connector to allow the rats to move freely in the cage. Food (pellets, pieces of fruit, and fruit loops) was distributed throughout the cage floor to encourage movement and exploration. Video data were recorded using a camcorder with a series of IR LEDs to enable recording in the dark, i.e., the active period for the rats. EMG data were amplified and recorded using custom LabView-based data acquisition software with a sampling frequency of 10 kHz. Data were recorded for 6 continuous hours starting at 8:00 μm and ending at 2:00 am. EMG recordings from the hindlimb muscles were bandpass filtered (1 Hz to 5 KHz), amplified using an A-M Systems Model 1700 differential AC amplifier (A-M Systems, Carlsborg, Wash.), and sampled at a frequency of 10 KHz using a custom data acquisition program written in the LabView development environment (National Instruments, Austin, Tex.).

### Data Analysis

The energy in the EMG signal for both muscles was calculated by estimating the area under the curve after rectification of the raw EMG (integrated EMG). The amounts of integrated EMG per one-min periods of stepping and spontaneous cage activity were compared. The EMG responses during spontaneous cage activity were binned in 1-min snippets for detailed analysis. A frequency distribution was constructed by estimating the energy within each 1-min bin and joint probability distributions to show the relationship between the activity of the soleus and TA were plotted. Video data were analyzed to estimate the total amount of time that the rats were active (mobile) in their home cages during the 6-hr recording period.

### Statistical Analyses

All data are reported as mean±SEM. Statistically significant differences were determined using paired t-tests. The criterion level for the determination of a statistical difference was set at P<0.05 for all computations.

### Results

### Evidence of Enabling Vs. Inducement of Neuromuscular Activity

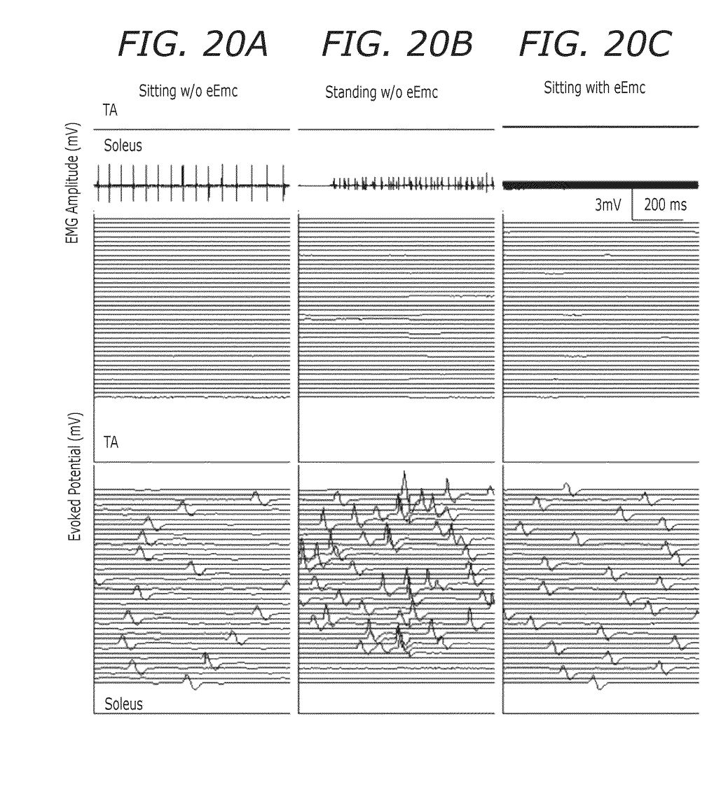

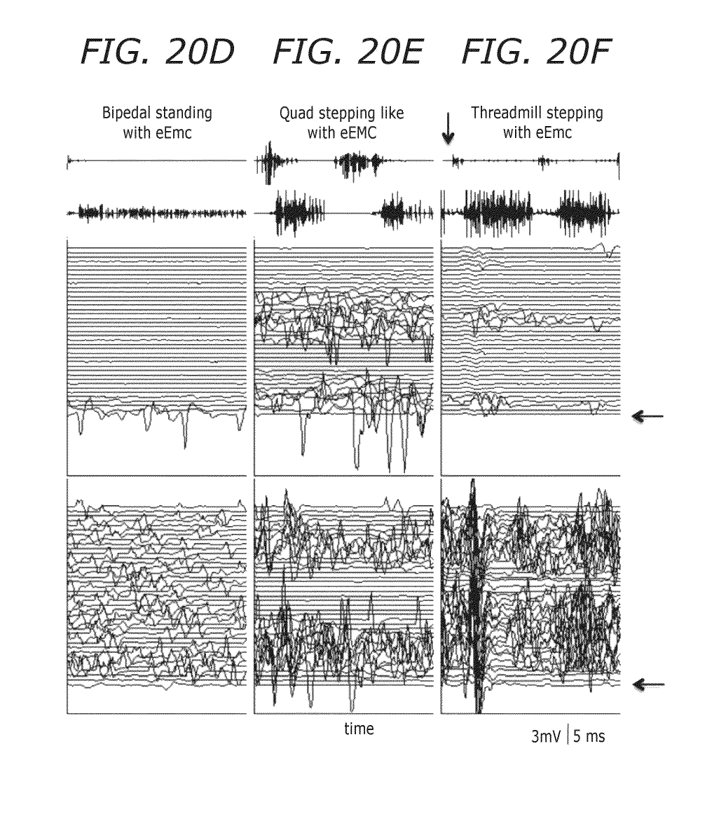

We examined the relationship between the absence or presence of eEmc and the amount and pattern of spontaneous cage activity. In the absence of eEmc there were periods of spontaneous activity when the rats remained in a sitting posture (FIG. 20A) and on some occasions when it appeared that they were attempting to stand (FIG. 20B). EMG activity increased, particularly in the soleus, during incidences of apparent attempted standing (FIG. 20B). The most common observed position was for the rats to have their hindlimbs completely extended often showing little or no movement except some spastic-like reactions. Even during movement propelled by the forelimbs, the upper body remained low with the head close to the floor of the cage and the hindlimbs extended.

A sub-motor threshold intensity of eEmc is evident by the absence of any time-linked evoked muscle responses (FIG. 20C). In the presence of eEmc the forelimbs were used to move around in the cage more often than in its absence.

During this activity the hindlimbs usually dragged behind showing some bursting in both the flexor and extensor muscles (FIG. 20E) and the upper body was maintained at a greater height compared with that seen without eEmc. The rats often would stand on the hindlimbs with partial weight bearing using the sides of the cage as support (FIG. 20D), a behavior never observed without eEmc.

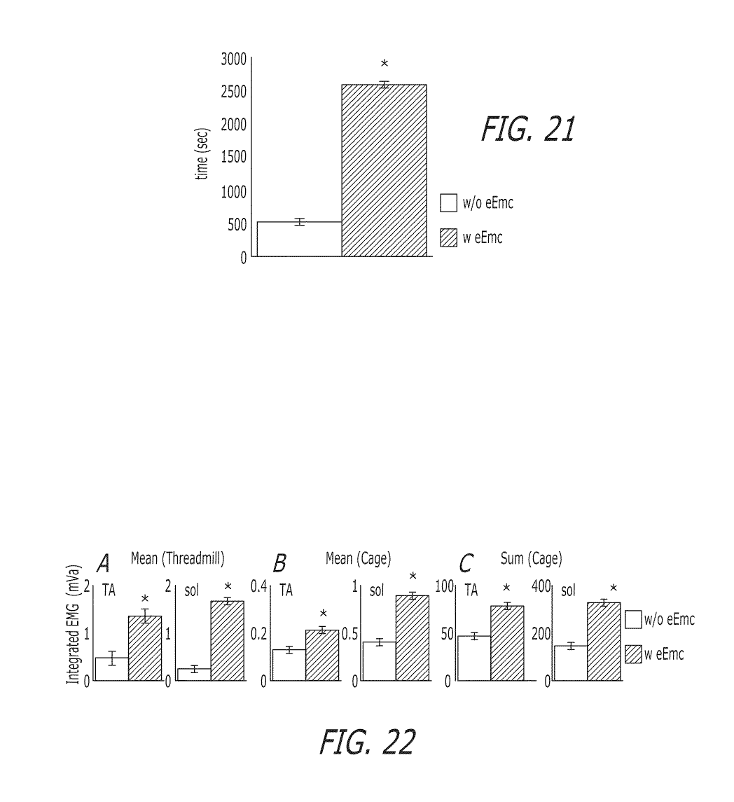

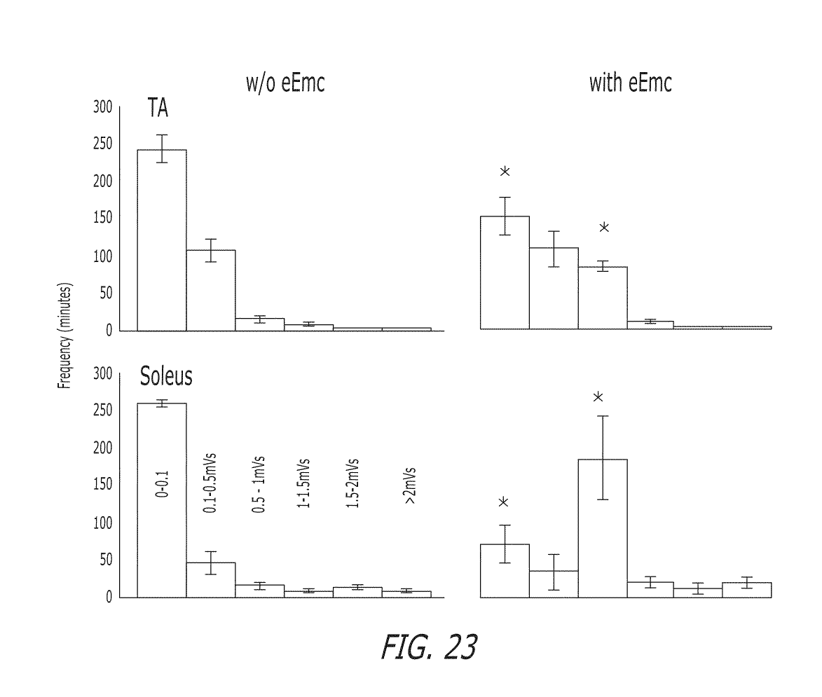

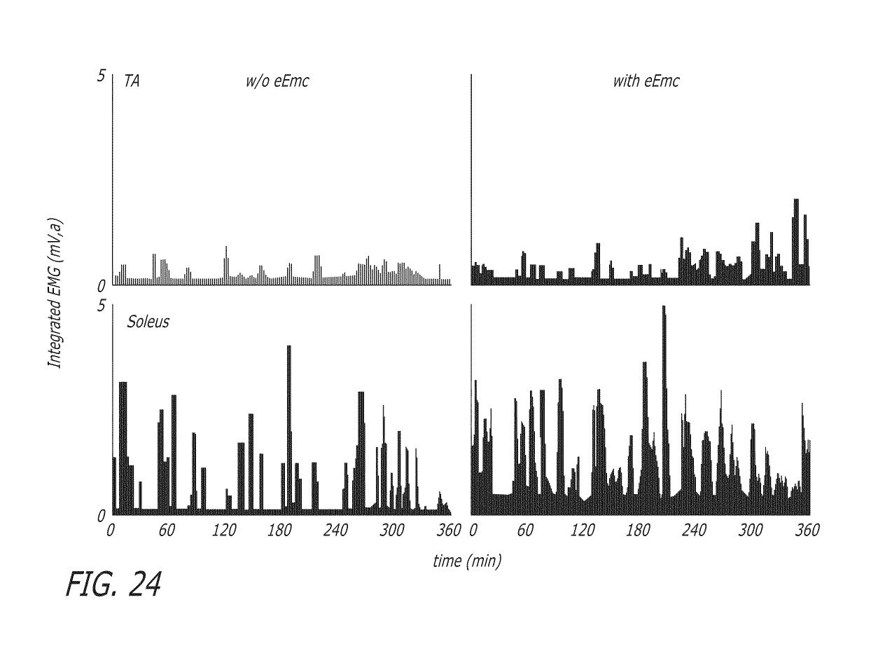

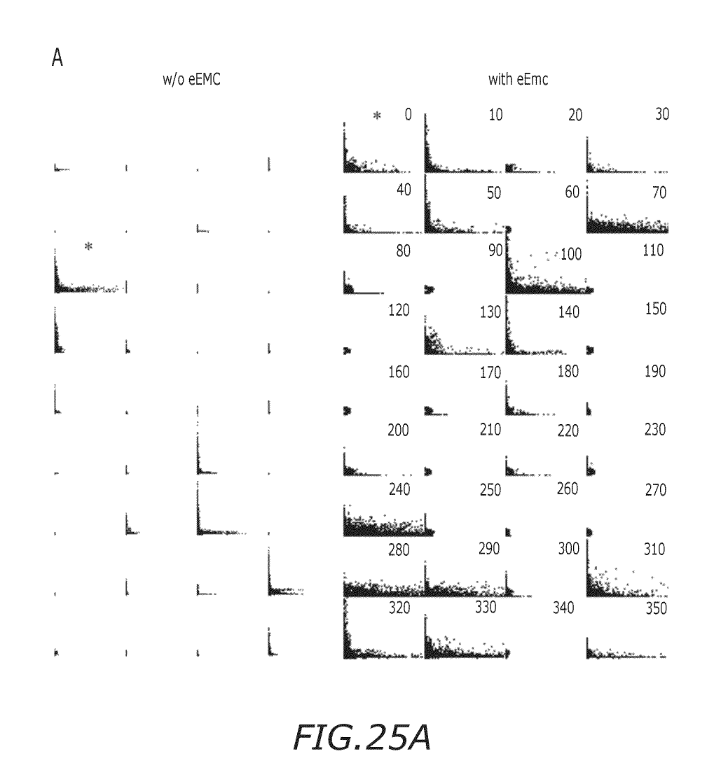

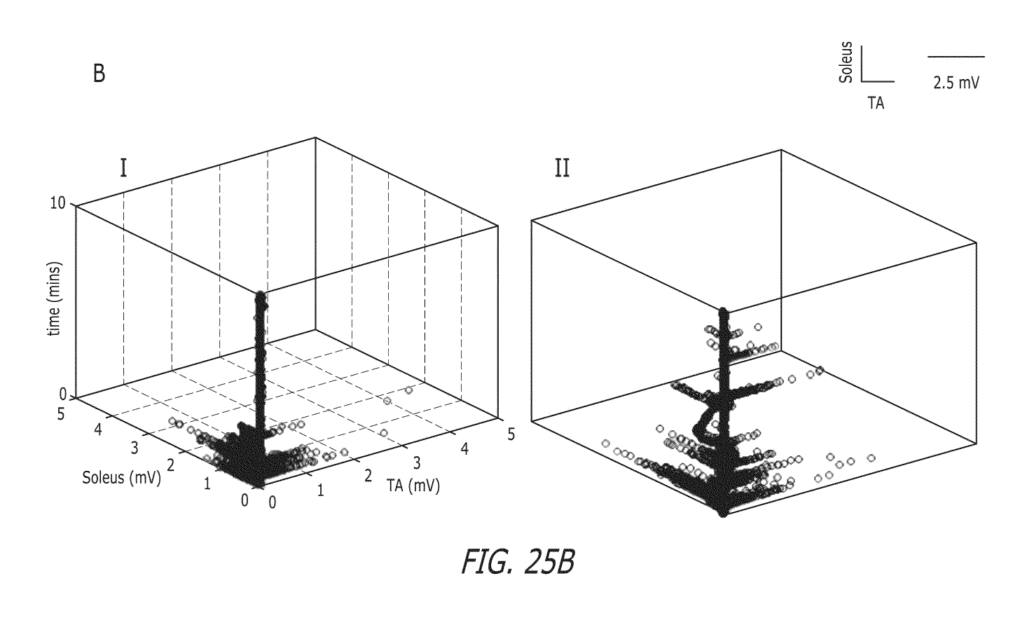

We compared the pattern of EMG activity during step-like movements generated spontaneously in the cage (FIG. 20E) to when the rat was stepping bipedally on a treadmill using the body weight support system (FIG. 20F). Although the stimuli imposed did not induce synchronized (time locked to stimulation pulses) motor responses with each individual stimulus (FIG. 20C), it was sufficient to enable a higher level of EMG activity in the TA and soleus and to produce motor responses that were asynchronous (not time locked to stimulation pulses) as occurs in the intact state (FIGS. 20D and E). Also, there is a greater level of synchronous activity during treadmill stepping (FIG. 20F) than during spontaneous cage activity (FIG. 20E). The total amount of time that the rats were active during these recordings was ˜5-fold higher in the presence compared to the absence of eEmc, i.e., ˜2500 sec or ˜12% of the time vs. ˜500 sec or ˜2.5% of the time (FIG. 21). The mean integrated EMG (FIG. 22B) and summed integrated EMG (FIG. 22C) for both the TA and soleus muscles during the 6-hr recording periods of spontaneous cage activity were significantly higher in the presence than in the absence of eEmc. To provide some point of reference regarding these increases in EMG activity with stimulation, the large differences in the mean integrated EMG in both muscles studied with and without eEmc when the rats were stepping on a treadmill are shown (FIG. 22A). Furthermore, the amount of activity during the six hours of spontaneous cage activity was equivalent to ˜33 minutes of stepping on the treadmill with eEmc compared to ˜15 minutes without stimulation. There was a larger number of one-min bins with relatively high levels of integrated EMG activity with than without eEmc distributed across the 6-hr recording period for both the TA and soleus (FIG. 23). Differences in the frequency distributions of EMG amplitudes with and without eEmc also were evident (FIG. 24). Higher EMG amplitudes were observed more frequently in both the TA and soleus in the presence of eEmc. There was greater evidence of reciprocal coordination between the TA and soleus muscles with than without eEmc across the 6-hr recording period (FIG. 25A). The level of EMG amplitude modulation was greater in the TA than the soleus and with this increased occurrence of higher amplitudes in the TA there was clearly a higher incidence of co-contraction between the TA and soleus muscles without eEmc. In addition, instances showing apparent reciprocal activity without eEmc had fewer and less robust alternating patterns (FIG. 25B I) compared to those observed with eEmc (FIG. 25B II).

### Discussion

Spinal circuits controlling stepping and standing after a spinal cord injury can be improved by practicing those tasks, i.e., increasing the activation of those circuits. Here, it is shown that there can be a minimal amount of spontaneous activity in the sensorimotor circuits that can facilitate standing and stepping after a midthoracic spinal cord transection in adult rats. eEmc below the level of the lesion, enhanced the amount of spontaneous activity several-fold (FIG. 22 and FIG. 24) and resulted in more robust stepping-like and partial weightbearing standing activity (FIGS. 20D and E). Thus, spontaneous activity can be enhanced with eEmcto provide a ‘self-training’ phenomenon, and independent, full weight-bearing standing can be initiated “voluntarily” and sustained in humans with complete paralysis in the presence of eEmc at an intensity that, in itself, induces little or no direct motor responses.

In some embodiments, elevated motor activity observed with subthreshold spinal cord stimulation reflects some level of voluntary control. These voluntary movements are not or may not be reflexes. While there are no widely accepted criteria for describing if a task is performed voluntarily, human subjects can acquire the ability to initiate and sustain standing on command. Subjects undergoing eEmc are able to volitionally position the upper body in a manner that increased weight bearing on the lower limbs with a critical level of eEmc. This, in turn, engaged the proprioceptive input to the spinal cord from the hindlimbs resulting in more weight-bearing activity, essentially as it seems to have been the case in the rats.

In other embodiments, the delivery of eEmc described herein can provide an individual with the ability to routinely and voluntarily engage proprioception to perform a motor task. The observations in the spinal rats in the present study can parallel human data in that the rats increased their cage activity levels in the presence of sub-motor threshold stimulation intensities (FIGS. 21, 23 and 24). They were more active and mobile because the spinal networks were placed in a state of higher “readiness”, making it more feasible to volitionally engage the postural and locomotor circuits when the rat chose to be mobile. In some embodiments, given that proprioception can initiate and control a wide range of postural and locomotor tasks, the elevated activity in the presence of eEmc occurred as a result of the intent of the rats to be mobile.

The experiments were designed to engage the paralyzed circuits during a specific training-rehabilitation time period in the presence of stimulation. Since the level of stimulation necessary to achieve the results mauy have had little or no recognizable direct motor or behavioral effects on the animal or human subjects, we tested whether sustained sub-threshold levels of activity in the normal cage environment would result in greater spontaneous activity among those spinal circuits that generate and control standing and stepping in rats. The observations of this testing provides that the training effects induced via formal motor rehabilitation sessions may be greatly amplified during periods of routine daily activity enhanced by eEmc.

In some embodiments, a general increase in activity may result in improved standing and stepping ability compared to rats not stimulated. In other embodiments, the improvement can be a result of the specificity of training. Further, rats that are housed in an enriched environment after a spinal cord injury are more active and perform significantly better in reaching and locomotor tasks than those housed in standard cages. Even further still, other stimulation paradigms may produce more robust task-specific effects.

The spinal rats in the present study were more spontaneously active with than without eEmc, even though they were housed in standard cages. In some embodiments, a combination of eEmc and an enriched housing environment may result in greater levels of spontaneous activity, particularly in rats that are completely paralyzed. The type and intensity of the activity performed by a spinal cord injured patient (or animal) during the prolonged daily periods without any formal rehabilitation treatment (most likely >23 hrs) can vary. In normal humans and animals 80-90% of the daily activity can occur at very low levels of activation of almost all motor pools. Further, daily activity levels can change when uninjured individuals begin physical training.

Epidural stimulation alone or in combination with an enriched environment may result in improved performance of reaching, standing, and locomotion and how much and what type of spontaneous activity is sufficient to enhance each of these motor tasks. Motor performance can be improved after severe paralysis by enabling the spinal circuitry during routine daily activities in the home in addition to the specific training that occurs during structured rehabilitation sessions. The spontaneous activity that may occur in a wide range of sensorimotor pathways may result in progressive improvement in specific tasks requiring fine motor control of the hands or in postural and locomotor functions, particularly if the same motor pathways are engaged as they are during scheduled rehabilitative sessions.

In some embodiments, there is an enhanced amount of spontaneous activity in the sensorimotor circuits that can facilitate standing and stepping after a mid-thoracic spinal cord transection in adult rats using chronic subthreshold eEmc below the level of the lesion.

1.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entireties for all purposes.

The foregoing described embodiments depict different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. Furthermore, it is to be understood that the invention is solely defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

Accordingly, the invention is not limited except as by the appended claims.

We claim:

1. A method comprising:
administering to a mammal with a paralysis an electrical enabling motor control stimulation to at a sub-threshold location, wherein the electrical enabling motor control stimulation provides spontaneous voluntary movement of at least one body part.

2. The method of claim 1, wherein the at least one body part is a toe, ankle, leg, knee, finger, elbow, shoulder, hand, hip, chest, trunk, neck, or a combination thereof.

3. The method of claim 1, wherein the paralysis is a motor complete paralysis.

4. The method of claim 1, wherein the paralysis is a motor incomplete paralysis.

5. The method of claim 1, wherein the paralysis was caused by a spinal cord injury classified as motor complete.

6. The method of claim 1, wherein the paralysis was caused by a spinal cord injury classified as motor incomplete.

7. The method of claim 1, wherein the paralysis was caused by a neurodegenerative brain injury.

8. The method of claim 7, wherein the neurodegenerative brain injury is associated with at least one of Parkinson's disease, Huntington's disease, Alzheimer's, ischemia, stroke, amyotrophic lateral sclerosis (ALS), primary lateral sclerosis (PLS), and cerebral palsy.

9. The method of claim 1, wherein the electrical enabling motor control stimulation is applied by one or more electrode or electrode array that is implanted epidurally in the spinal cord of the mammal.

10. The method of claim 1, further comprising:
administering one or more neuropharmaceutical agents to the mammal.

11. The method of claim 10, wherein the one or more neuropharmaceutical agents comprise at least one of a serotonergic drug, a dopaminergic drug, a noradrenergic drug, a GABAergic drug, and glycinergic drugs.

12. The method of claim 10, wherein the one or more neuropharmaceutical agents comprise at least one of 8-OHDPAT, Way 100.635, Quipazine, Ketanserin, SR 57227A, Ondanesetron, SB 269970, Methoxamine, Prazosin, Clonidine, Yohimbine, SKF-81297, SCH-23390, Quinpirole, Busiprone, and Eticlopride.

13. The method of claim 1, wherein the administration of the electrical enabling motor control stimulation makes the mammal at least 2 times more active than without the electrical enabling motor control stimulation.

14. The method of claim 1, wherein the administration of the electrical enabling motor control stimulation makes the mammal at least 5 times more active than without the electrical enabling motor control stimulation.

15. The method of claim 1, wherein the spontaneous voluntary movement is a stepping-like activity, a partial weight bearing standing activity, a reaching activity, a grasping activity, a pulling activity, a pushing activity, extending activity, a flexing activity, a rotating activity, or a movement of a erect penis.

16. The method of claim 1, wherein the electrical enabling motor control stimulation is applied by one or more electrode or electrode array that is applied transcutaneously over a region of the spinal cord of the mammal.

17. The method of claim 1, further comprising:
administering the electrical enabling motor control stimulation in a positive environment.

18. The method of claim 17, wherein the positive environment is a location familiar to the mammal.

19. The method of claim 17, wherein the positive environment assists in attaining spontaneous voluntary movement.

20. The method of claim 1, further comprising:
Administering the electrical enabling motor control stimulation based on at least one sensory input.
